(19) United States
(12) Patent Application Publication
(10) Pub. No.: US 2024/0417900 A1
(43) Pub. Date: Dec. 19, 2024

(54) NONWOVEN FABRIC HAVING IMPROVED SOFTNESS

(71) Applicant: FITESA SIMPSONVILLE, INC., Simpsonville, SC (US)

(72) Inventors: Thitaree KONGDATE, Rayong (TH); Jirawan JIRAPONG, Rayong (TH)

(73) Assignee: FITESA SIMPSONVILLE, INC., Simpsonville, SC (US)

(21) Appl. No.: 18/663,674

(22) Filed: May 14, 2024

Related U.S. Application Data
(60) Provisional application No. 63/521,466, filed on Jun. 16, 2023.

(51) Int. Cl. D04H 1/4291; A61F 13/15; A61L 15/22; A61L 15/42; D04H 1/4374; D04H 1/4382; D04H 1/559; D04H 1/56

(52) U.S. Cl. CPC D04H 1/4291; A61F 13/15699; A61L 15/225; A61L 15/42; D04H 1/4374; D04H 1/43828; D04H 1/43838; D04H 1/559; D04H 1/56; A61F 2013/15447; A61F 2013/15959; A61F 2013/15967; A61F 2013/15991; D10B 2321/022; D10B 2401/063; D10B 2509/026

(57) ABSTRACT

A nonwoven fabric having a plurality of fibers bonded to form a coherent web, the fibers are formed of a polymeric blend of a polypropylene resin and an elastomeric polyolefin, wherein the fabric exhibits a decrease in fiber fineness of at least 5% in comparison to an identically prepared nonwoven fabric that does not include the elastomeric polyolefin blended with the polypropylene resin.

### CROSS REFERENCE TO RELATED APPLICATIONS

The present application claims the benefit of U.S. Provisional Application 63/521,466, filed Jun. 16, 2023, the entire contents of which are hereby incorporated by reference.

### FIELD

The presently-disclosed invention relates generally to nonwoven fabrics, and more particularly to bonded nonwoven fabrics exhibiting improvements in fiber fineness and softness.

### BACKGROUND

Nonwoven fabrics are used in a variety of applications such as garments, disposable medical products, and absorbent articles such as diapers and personal hygiene products, among others. New products being developed for these applications have demanding performance requirements, including comfort, conformability to the body, freedom of body movement, good softness and drape, adequate tensile strength and durability, and resistance to surface abrasion, pilling or fuzzing. Accordingly, the nonwoven fabrics which are used in these types of products must be engineered to meet these performance requirements.

Despite significant efforts in developing nonwoven fabrics, there is still a need for products exhibiting improvements in softness and fiber fineness without sacrificing other beneficial properties such as mechanical properties.

### SUMMARY

One or more embodiments of the invention may provide a nonwoven fabric having desirable properties with respect to fiber fineness and softness while maintaining good mechanical properties, such as tensile strength and elongation.

Certain embodiments are directed to a nonwoven fabric comprising a plurality of fibers bonded with a bond pattern on a surface thereof to form a coherent web in which the nonwoven fabric.

In certain embodiments, a nonwoven fabric is provided in which the fabric comprises a plurality of fibers bonded to form a coherent web, and in which the fibers comprise a polymeric blend of a polypropylene resin and an elastomeric polyolefin, and wherein the fabric exhibits a decrease in fiber fineness of at least 5% in comparison to an identically prepared nonwoven fabric that does not include the elastomeric polyolefin blended with the polypropylene resin.

In certain embodiments, the polypropylene resin has a molecular weight ranging from any of 120,000 to 300,000 g/mol, 140,000 g/mol to about 280,000 g/mol, from about 150,000 to about 250,000 g/mol, and in particular, from about 160,000 to about 180,000 g/mol. In some embodiments, the polypropylene resin comprises a Ziegler-Natta catalyzed polypropylene, a metallocene catalyzed polypropylene, or a blend thereof.

In certain embodiments, the fibers are bicomponent fibers having a sheath core configuration in which the elastomeric polyolefin is only present in the sheath or the core. In other embodiments, the elastomeric polyolefin may be present in both the sheath or the core.

In certain embodiments, the polypropylene resin of the blend has a melting temperature from about 150° C. to about 175° C. In certain embodiments, the polypropylene resin is present in the polymer blend in an amount that is from about 75 to 99 weight percent, based on the total weight of polymer blend, and in particular, from about 80 to 95 weight percent, and more particularly, from about 85 to 94 weight percent, based on the total weight of the polymer blend.

In certain embodiments, the elastomeric polyolefin is present in the polymer blend in an amount ranging from about 2 to 30 weight percent, and in particular, 5 to 25 weight percent, and more particularly, from about 8 to 20 weight percent, based on the total weight of the polymer blend.

In certain embodiments, the elastomeric polyolefin comprises a propylene-alpha-olefin copolymer.

In certain embodiments, the elastomeric polyolefin comprises a low isotacticity polypropylene polymer.

In certain embodiments, the low isotacticity polypropylene has an isotacticity [mmmm] that is between about 20 and 70% by mol, and in particular, a [mmmm] between 30 and 60% by mol, and more particularly, a [mmmm] between 35 and 55% by mol.

In certain embodiments, the low isotacticity polypropylene has the following properties:
an isotacticity: a meso pentad fraction [mmmm] of 20 to 70% by mol;
an average number molecular weight (Mw) of 10,000 to 200,000;
a melting temperature from about 60 to 120° C.; and
a melt flow rate (MFR) greater than 40 g/10 min.

In certain embodiments, the nonwoven fabric comprises fibers having an average fiber fineness ranging from about 0.8 to 1.6 dtex, and in particular, from about 0.9 to 1.4. and more particularly, from about 1.2 to 1.35 dtex.

In certain embodiments, the nonwoven fabric exhibits a percent decrease in fiber dtex from about 5 to 30% in comparison to an identically prepared fabric with the exception that the identically prepared fabric does not include the elastomeric polyolefin and is drawn and attenuated at a cabin pressure of less than 4,200 Pa.

In certain embodiments, the nonwoven fabric exhibits a percent decrease in fiber dtex from about 5 to 60% in comparison to an identically prepared fabric with the exception that the identically prepared fabric does not include the elastomeric polyolefin and in which the fibers are drawn and attenuated at a cabin pressure of less than 4,200 Pa.

In certain embodiments, the nonwoven fabric exhibits a percent decrease in fiber dtex from about 5 to 60%, such as a percent decrease of 15 to 50%, a percent decrease of 10 to 30%, and a percent decrease of 15 to 25% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has a machine direction bending softness ranging from 30 to 50 mm and a cross direction bending softness ranging from 15 to 40 mm.

In certain embodiments, the nonwoven fabric has an average machine/cross direction bending softness ranging from 20 to 45 mm, and in particular, from about 25 to 40 mm, an rom about 28 to 38 mm.

In certain embodiments, nonwoven fabric exhibits a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers are drawn and attenuated at a cabin pressure of less than 4,200 Pa.

In certain embodiments, the nonwoven fabric exhibits a percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers are drawn and attenuated at a cabin pressure of less than 4,200 Pa.

In certain embodiments, the nonwoven fabric exhibits one or more of the following characteristics:
a) an average fiber fineness ranging from about 0.8 to 1.6 dtex, and in particular, from about 0.9 to 1.4. and more particularly, from about 1.2 to 1.35 dtex;
b) the nonwoven fabric exhibits a percent decrease in fiber dtex from about 5 to 60%, such as a percent decrease of 15 to 25%, in comparison to an identically prepared fabric with the exception that the identically prepared fabric does not include the elastomeric polyolefin and in which the fibers were drawn and attenuated at a cabin pressure of less than 4,200 Pa;
c) the nonwoven fabric exhibits a machine direction bending softness ranging from 30 to 50 mm direction and a cross direction bending softness ranging from 15 to 40 mm
d) the nonwoven fabric exhibits a machine direction softness of less than one or more of 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, and 20 mm;
e) a machine direction softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, and 46 mm;
f) an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.
g) a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers were drawn and attenuated at a cabin pressure of less than 4,200 Pa;
h) an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers were drawn and attenuated at a cabin pressure of less than 4,200 Pa;
i) an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the fibers were drawn and attenuated at a cabin pressure of less than 4,200 Pa;
j) an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the fibers were drawn and attenuated at a cabin pressure of less than 4,200 Pa;
k) an increase in MD and CD tensile strengths ranging from about 2 to 15% in comparison to an identically prepared fabric with the exception that the identically prepared fabric does not include the elastomeric polyolefin and in which the fibers were drawn and attenuated at a cabin pressure of less than 4,200 Pa; and
l) an increase in percent elongation in one or more of machine or cross direction ranging from about 5 to 30% in comparison to an identically prepared fabric with the exception that the identically prepared fabric does not include the elastomeric polyolefin and in which the fibers were drawn and attenuated at a cabin pressure of less than 4,200 Pa.

In certain embodiments, the nonwoven fabric has an average fiber fineness ranging from about 0.8 to 1.6 dtex, and in particular, from about 0.9 to 1.4. and more particularly, from about 1.2 to 1.35 dtex.

In certain embodiments, the nonwoven fabric has a percent decrease in fiber dtex from about 5 to 30%, such as a percent decrease of 15 to 25%, in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an a machine direction bending softness ranging from 30 to 50 mm direction and a cross direction bending softness ranging from 15 to 40 mm.

In certain embodiments, nonwoven fabric has an a machine direction softness of less than one or more of 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, and 20 mm.

In certain embodiments, the nonwoven fabric has an a machine direction bending softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, 46 mm, 48 mm, and 50 mm.

In certain embodiments, the nonwoven fabric has an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.

In certain embodiments, the nonwoven fabric has an a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an average percent decrease in machine/cross direction bending softness that is from about 20 to 40%, and in particular, from about 20 to 35%, and wherein more particularly, from about 22 to 30% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an increase in MD and CD tensile strengths ranging from about 2 to 15% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an increase in percent elongations in one or more of machine or cross direction ranging from about 5 to 30% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has the following properties:
an average fiber fineness ranging from about 0.8 to 1.6 dtex, and in particular, from about 0.9 to 1.4. and more particularly, from about 1.2 to 1.35 dtex;
a percent decrease in fiber dtex from about 5 to 60%, such as a percent decrease of 15 to 25%, in comparison to an identically prepared fabric with the exception that the identically prepared fabric does not include the elastomeric polyolefin and in which the filaments were drawn and attenuated at a cabin pressure of less than 4,200 Pa; and
the nonwoven fabric exhibits a machine direction bending softness ranging from 30 to 50 mm direction and a cross direction bending softness ranging from 15 to 40 mm, such as a machine direction bending softness ranging from 35 to 45 mm direction and a cross direction bending softness ranging from 25 to 40 mm.

In certain embodiments, the nonwoven fabric comprises a spunbond layer.

In certain embodiments, the nonwoven fabric comprises a first spunbond layer having low or no crimping filaments and a second layer comprising crimped filaments.

In certain embodiments, the nonwoven fabric comprises at least two layers in which one of the layers is selected from the group of meltblown layer; carded fabric layer, spunbond layer, resin bonded layer, airlaid fabric layer, and a spunlace layer.

In certain embodiments, the nonwoven fabric is in an absorbent article.

In a further aspect, embodiments of the invention provide a nonwoven articles comprising a nonwoven fabric in accordance with embodiments of the invention.

In certain embodiments, a composite sheet material comprises one or more nonwoven fabric layers comprising the nonwoven fabric in accordance with embodiments of the invention, such as a sheet material comprising a meltblown layer, the sheet material comprises a meltblown layer.

In certain embodiments, the composite sheet material comprises one or more meltblown layers sandwiched between two spunbond layers.

Certain aspects are directed to an absorbent article comprising the nonwoven fabric in accordance with one or more embodiments of the invention.

Aspects of the invention are also directed to a method of preparing a bonded nonwoven fabric comprising:
melt blending a first polypropylene resin and an elastomeric polyolefin to form a molten or semi-molten polymer stream of a polymer blend;
introducing the polymer stream into a spinning beam;
extruding the polymer stream from the spinning beam to form fibers;
subjecting the formed fibers to a cabin pressure greater than 4,200 Pa to draw and attenuate the fibers; and
collecting the fibers on a collection surface to form a nonwoven web, wherein the an average fiber fineness ranging from about 0.8 to 1.6 dtex, and in particular, from about 0.9 to 1.4. and more particularly, from about 1.2 to 1.35 dtex, and the nonwoven fabric exhibits a percent decrease in fiber dtex from about 5 to 30%, such as a percent decrease of 15 to 25%, in comparison to an identically prepared fabric with the exception that the identically prepared fabric does not include the elastomeric polyolefin and in which the fibers were drawn and attenuated at a cabin pressure of less than 4,200 Pa.

In certain embodiments, the cabin pressure ranges from about 4,500 to 7,500 Pa, such as from 4,800 to 7,500, 5,000 to 6,000, and 5,100 to 5,600 Pa.

In certain embodiments, the blend and components thereof are in accordance with the compositions previously described.

Aspects of the nonwoven fabric and associated method are also directed to the use of such to prepare an absorbent article.

In further aspect, the method also comprises a step of depositing a second fabric layer over the nonwoven web. In certain embodiment, the second fabric layer is selected from the group of meltblown layer; carded fabric layer, spunbond layer, resin bonded layer, airlaid fabric layer, and a spunlace layer.

In certain embodiments, the method further comprises a step of thermal bonding the nonwoven web.

In certain embodiments, the step of thermal bonding the nonwoven web comprises calender bonding the nonwoven web with an engraved roll having raised bonding points configured and arranged to impart a bonding pattern on a surface of the nonwoven web, the bonding pattern having a percent bonded area from about 9.6 to 14%, an average individual bond surface area from about 0.10 to 0.25 mm², and an average bond point packing value from about 4 to 7.25 mm⁻¹.

### BRIEF DESCRIPTION OF THE SEVERAL DRAWINGS

Having thus described the invention in general terms, reference will now be made to the accompanying drawings, which are not necessarily drawn to scale, and wherein:

FIG. 1 illustrates a nonwoven fabric in accordance with at least one embodiment of the invention;

FIG. 2 illustrates a system for preparing a nonwoven fabric in accordance with at least one embodiment of the present invention;

FIG. 3 illustrates a system for preparing a nonwoven fabric in accordance with at least one embodiment of the present invention;

FIGS. 4A-4D illustrate multilayer nonwoven fabrics in accordance with at least one embodiment of the present invention; and

FIG. 5 shows a general bonding pattern used in point bonding nonwoven fabrics in accordance with embodiments of the invention.

### DETAILED DESCRIPTION

The invention now will be described more fully hereinafter with reference to the accompanying drawings, in which some, but not all embodiments of the inventions are shown. Indeed, this inventions may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

The terms “first,” “second,” and the like, “primary,” “exemplary,” “secondary,” and the like, do not denote any order, quantity, or importance, but rather are used to distinguish one element from another. Further, the terms “a,” “an,” and “the” do not denote a limitation of quantity, but rather denote the presence of “at least one” of the referenced item.

Each embodiment disclosed herein is contemplated as being applicable to each of the other disclosed embodiments. All combinations and sub-combinations of the various elements described herein are within the scope of the invention.

It is understood that where a parameter range is provided, all integers within that range, and tenths and hundredths thereof, are also provided by the invention. For example, “5-10%” includes 5%, 6%, 7%, 8%, 9%, and 10%; 5.0%, 5.1%, 5.2% . . . 9.8%, 9.9%, and 10.0%; and 5.00%, 5.01%, 5.02% . . . 9.98%, 9.99%, and 10.00%.

As used herein, the terms “about.” “approximately,” and “substantially” in the context of a numerical value or range means±10% of the numerical value or range recited or claimed, and in particular, encompasses values within a standard margin of error of measurement (e.g., SEM) of a stated value or variations ±0.55%, 1%, 5%, or 10% from a specified value.

For the purposes of the present application, the following terms shall have the following meanings:

The term “fiber” can refer to a fiber of finite length or a filament of infinite length.

As used herein, the term “monocomponent” refers to fibers formed from one polymer or formed from a single blend of polymers. Of course, this does not exclude fibers to which additives have been added for color, anti-static properties, lubrication, hydrophilicity, liquid repellency, etc.

As used herein, the term “multicomponent” refers to fibers formed from at least two polymers (e.g., bicomponent fibers) that are extruded from separate extruders. The at least two polymers can each independently be the same or different from each other, or be a blend of polymers. The polymers are arranged in substantially constantly positioned distinct zones across the cross-section of the fibers. The components may be arranged in any desired configuration, such as sheath-core, side-by-side, pie, island-in-the-sea, and so forth. Various methods for forming multicomponent fibers are described in U.S. Pat. No. 4,789,592 to Taniguchi et al. and U.S. Pat. No. 5,336,552 to Strack et al., U.S. Pat. No. 5,108,820 to Kaneko, et al., U.S. Pat. No. 4,795,668 to Kruege, et al., U.S. Pat. No. 5,382,400 to Pike, et al., U.S. Pat. No. 5,336,552 to Strack, et al., and U.S. Pat. No. 6,200,669 to Marmon, et al., which are incorporated herein in their entirety by reference. Multicomponent fibers having various irregular shapes may also be formed, such as described in U.S. Pat. No. 5,277,976 to Hogle, et al., U.S. Pat. No. 5,162,074 to Hills, U.S. Pat. No. 5,466,410 to Hills, U.S. Pat. No. 5,069,970 to Largman, et al., and U.S. Pat. No. 5,057,368 to Largman, et al., which are incorporated herein in their entirety by reference.

As used herein the terms “nonwoven,” “nonwoven web” and “nonwoven fabric” refer to a structure or a web of material which has been formed without use of weaving or knitting processes to produce a structure of individual fibers or threads which are intermeshed, but not in an identifiable, repeating manner. Nonwoven webs have been, in the past, formed by a variety of conventional processes such as, for example, meltblown processes, spunbond processes, and staple fiber carding processes.

As used herein, the term “meltblown” refers to a process in which fibers are formed by extruding a molten thermoplastic material through a plurality of fine, usually circular, die capillaries into a high velocity gas (e.g. air) stream which attenuates the molten thermoplastic material and forms fibers, which can be to microfiber diameter. Thereafter, the meltblown fibers are carried by the gas stream and are deposited on a collecting surface to form a web of random meltblown fibers. Such a process is disclosed, for example, in U.S. Pat. No. 3,849,241 to Buntin et al.

As used herein, the term “machine direction” or “MD” refers to the direction of travel of the nonwoven web during manufacturing.

As used herein, the term “cross direction” or “CD” refers to a direction that is perpendicular to the machine direction and extends laterally across the width of the nonwoven web.

As used herein, the term “diagonal direction” or “DD” refers to a direction that is angled from greater than 0° to less than 90° relative to one or more of the cross and machine directions.

As used herein, and unless indicated to the contrary, the term “molecular weight” refers to the weight average molecular weight (Mw), and is expressed in grams/mol. The weight average molecular weight can be determined using commonly known techniques, such as gel permeation chromatography (GPC).

As used herein, the term “spunbond” refers to a process involving extruding a molten thermoplastic material as filaments from a plurality of fine, usually circular, capillaries of a spinneret, with the filaments then being attenuated and drawn mechanically or pneumatically. The filaments are deposited on a collecting surface to form a web of randomly arranged substantially continuous filaments which can thereafter be bonded together to form a coherent nonwoven fabric. The production of spunbond non-woven webs is illustrated in patents such as, for example, U.S. Pat. Nos. 3,338,992; 3,692,613, 3,802,817; 4,405,297 and 5,665,300. In general, these spunbond processes include extruding the filaments from a spinneret, quenching the filaments with a flow of air to hasten the solidification of the molten filaments, attenuating the filaments by applying a draw tension, either by pneumatically entraining the filaments in an air stream or mechanically by wrapping them around mechanical draw rolls, depositing the drawn filaments onto a foraminous collection surface to form a web, and bonding the web of loose filaments into a nonwoven fabric. The bonding can be any thermal or chemical bonding treatment, with thermal point bonding being typical.

As used herein “thermal point bonding” involves passing a material such as one or more webs of fibers to be bonded between a heated calender roll and an anvil roll. The calender roll is typically engraved with a pattern so that the fabric is bonded in discrete point bond sites rather than being bonded across its entire surface.

As used herein, the term “air through thermal bonding” involves passing a material such as one or more webs of fibers to be bonded through a stream of heated gas, such as air, in which the temperature of the heated gas is above the softening or melting temperature of at least one polymer component of the material being bonded. Air through thermal bonding may involve passing a material through a heated oven.

As used herein, the term “bond density” refers to the number of individual bond points in a given surface area of the nonwoven fabric.

As used herein the term “polymer” generally includes, but is not limited to, homopolymers, copolymers, such as, for example, block, graft, random and alternating copolymers, terpolymers, etc. and blends and modifications thereof. Furthermore, unless otherwise specifically limited, the term “polymer” shall include all possible geometrical configurations of the material, including isotactic, syndiotactic and random symmetries.

### Nonwoven Fabric

Certain embodiments of the invention are directed to a nonwoven fabric exhibiting increased softness and fiber fineness in which at least some of the fibers of the nonwoven comprise a polymer blend of a polypropylene polymer and an elastomeric polyolefin polymer. In one embodiment, the present invention provides a nonwoven fabric comprising a plurality of fibers wherein the plurality fibers comprise a blend of a first polypropylene polymer and at least one elastomeric polyolefin polymer. As explained in greater detail below, the inclusion of the elastomeric polyolefin polymer in the polymer blend improves the softness and fineness of the fibers of the fabric in comparison to an identical fabric that does not include the elastomeric polyolefin polymer.

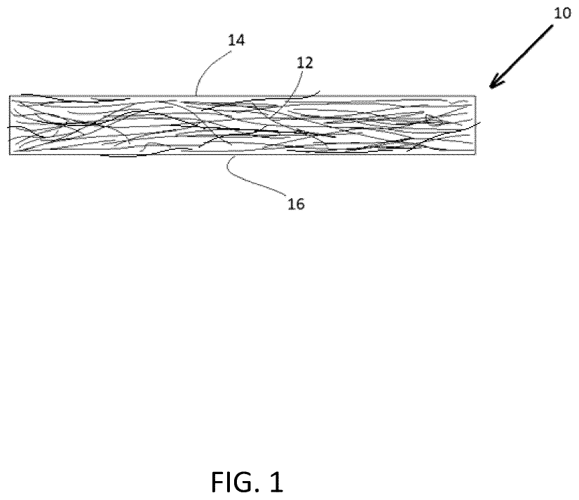

With reference to FIG. 1, a nonwoven fabric in accordance with at least one embodiment of the invention is shown and designated by reference character 10. The nonwoven fabric comprises a plurality of fibers 12 that are associated together to form a coherent web. The nonwoven fabric comprises a first outer surface 14 and a second outer surface 16. In some embodiments, the fibers of the nonwoven fabric are not subjected to a further bonding step and remain relatively unbonded.

Advantageously and surprisingly, the inventors of the present disclosure have discovered that improvements in both softness and fiber fineness may be obtained with nonwoven fabrics in which the fibers comprise a polymer blend comprising a first polypropylene polymer and an elastomeric polyolefin polymer, and in which the fibers are spun under certain spinning parameters as discussed in greater detail below.

### First Polypropylene Polymers

In certain embodiments, the first polypropylene polymer comprises the balance of the polymer blend and typically comprises a polypropylene polymer resin that is suitable for spinning continuous filaments in spunbond processes. In certain other embodiments, the first polypropylene polymer comprises a minor component of the polymer blend and the elastomeric polyolefin polymer comprises the balance of the polymer blend.

A wide variety of polypropylene polymers may be used in various embodiments of the present disclosure. Examples of suitable polypropylenes that may be used typically have molecular weights greater than about 120,000 g/mol, and more typically, 150,000 to about 300,000 g/mol. In one embodiment, the polypropylene polymer may have molecular weights ranging from about may have a molecular weights ranging from about 150,000 to about 250,000 g/mol, and in particular, from about 160,000 to about 180,000 g/mol. In certain embodiments, the polypropylene resin has a molecular weight ranging from any of 120,000 to 300,000 g/mol, 140,000 g/mol to about 280,000 g/mol, from about 150,000 to about 250,000 g/mol, and in particular, from about 160,000 to about 180,000 g/mol.

In certain embodiments for the preparation of spunbond fibers, suitable polypropylene resins typically have an MFR that is from about 10 to 100 g/10 min, and in particular, from about 20 to 40 g/10 min, with an MFR from about 22 to 38 g/10 min being somewhat more typical. Unless otherwise indicated MFR is measured in accordance with ASTM D-1238.

In certain embodiments, the polypropylene resin has a melting temperature from about 150° C. to about 175° C., such as a melting point ranging from about 150 to 160° C.

Examples of such polypropylenes may include those available from LyondellBasell under the product name Metocene HM562S (30 MFR g/10 min, density of 0.90 g/cm³); a Ziegler-Natta catalyzed homopolymer polypropylene available from IPRC Thailand under the product number 1105SC (35 MFR g/10 min.); ExxonMobil, such as PP3155 (36 MFR g/10 min, density of 0.90 g/cm³, and Mw 172 k g/mol); PP3155E5 (36 MFR g/10 min, density of 0.90 g/cm³, and Mw 172 k g/mol); and ACHIEVE™ 3854 (24 MFR g/10 min, density of 0.90 g/cm³). Polypropylenes available from SABIC®, such as SABIC PP 511A (25 MFR g/10 min, density of 0.905 g/cm³), polypropylenes available from Borealis, such as HG475FB (27 MFR g/10 min), and polypropylenes available from Braskem, such as CP360H (34 MFR g/10 min) may also be used.

The amount of the first polypropylene polymer in the polymer blend is typically from about 1 to 99 weight percent, and in particular, from about 5 to 95 weight percent, and more particularly, from about 10 to 90 weight percent, and even more particularly, from about 15 to 85 weight percent of the polymer blend, based on the total weight of the polymer blend.

In certain embodiments, the amount of the first polypropylene comprises from about 50 to 99 weight percent of the polymer blend, based on the total weight of the polymer blend. In certain preferred embodiments, the first polypropylene polymer comprises the balance of the polymer blend and as such, is present in an amount greater than 50 weight percent, based on the total weight of the blend. For example, the amount of the first polypropylene resin may be from about 65 to 99 weight percent, and in particular, from about 70 to 95 weight percent, and more particularly, from about 75 to 90 weight percent, based on the total weight of the polymer blend.

In certain embodiments, the amount of first polypropylene polymer resin in the polymer blend is at least 50 weight percent, at least 51 weight percent, at least 52 weight percent, at least 53 weight percent, at least 54 weight percent, at least 55 weight percent, at least 56 weight percent, at least 57 weight percent, at least 58 weight percent, at least 59 weight percent, at least 60 weight percent, at least 61 weight percent, at least 62 weight percent, at least 63 weight percent, at least 64 weight percent, 65 weight percent, at least 66 weight percent, at least 67 weight percent, at least 68 weight percent, at least 69 weight percent, at least 70 weight percent, at least 71 weight percent, at least 72 weight percent, at least 73 weight percent, at least 74 weight percent, at least 75 weight percent, at least 76 weight percent, at least 77 weight percent, at least 78 weight percent, at least 79 weight percent, at least 80 weight percent, at least 81 weight percent, at least 82 weight percent, at least 83 weight percent, at least 84 weight percent, at least 85 weight percent, at least 86 weight percent, at least 87 weight percent, at least 88 weight percent, at least 89 weight percent, at least 90 weight percent, at least 91 weight percent, at least 92 weight percent, at least 93 weight percent, at least 94 weight percent, at least 95 weight percent, at least 96 weight percent, at least 97 weight percent, at least 98 weight percent, and at least 89 weight percent, based on the total weight of the polymer blend.

In certain embodiments, the amount of first polypropylene polymer in the polymer blend is less than 99 weight percent, less than 98 weight percent, less than 97 weight percent, less than 96 weight percent, less than 95 weight percent, less than 94 weight percent, less than 93 weight percent, less than 92 weight percent, less than 91 weight percent, less than 90 weight percent, less than 89 weight percent, less than 88 weight percent, less than 87 weight percent, less than 86 weight percent, less than 85 weight percent, less than 84 weight percent, less than 83 weight percent, less than 82 weight percent, less than 81 weight percent, less than 80 weight percent, less than 79 weight percent, less than 78 weight percent, less than 77 weight percent, less than 76 weight percent, less than 75 weight percent, less than 74 weight percent, less than 73 weight percent, less than 72 weight percent, less than 71 weight percent, less than 70 weight percent, less than 69 weight percent, less than 68 weight percent, less than 67 weight percent, and less than 66 weight percent, less than 65 weight percent, less than 64 weight percent, less than 63 weight percent, less than 62 weight percent, less than 61 weight percent, less than 60 weight percent, less than 59 weight percent, less than 58 weight percent, less than 57 weight percent, less than 56 weight percent, less than 55 weight percent, less than 54 weight percent, less than 53 weight percent, less than 52 weight percent, and less than 51 weight percent based on the total weight of the polymer blend.

In certain embodiments, the polypropylene resin is present in the polymer blend in an amount that is from about 75 to 99 weight percent, based on the total weight of polymer blend, and in particular, from about 80 to 95 weight percent, and more particularly, from about 85 to 94 weight percent, based on the total weight of the polymer blend.

It should also be recognized that polymer blends in accordance with embodiments of the present disclosure include ranges of the polypropylene polymer resin between any of the aforementioned weight percentages, such as from about 50 to 99 weight percent, 60 to 98 weight percent, 67 to 97 weight percent, 68 to 96 weight percent, 69 to 96 weight percent, 70 to 95 weight percent, 71 to 94 weight percent, 72 to 93 weight percent, 73 to 92 weight percent, 74 to 91 weight percent, and 75 to 90 weight percent, and variations of these ranges, based on the total weight of the polymer blend.

In certain embodiments, the first polypropylene polymer resin comprises a metallocene catalyzed polypropylene. In certain embodiments, the first polypropylene polymer resin comprises a Zeigler-Natta catalyzed polypropylene. In certain embodiments, the first polypropylene polymer resin comprises a blend of a metallocene catalyzed polypropylene and a Zeigler-Natta catalyzed polypropylene.

When present as a blend of a metallocene catalyzed polypropylene and a Zeigler-Natta catalyzed polypropylene, the ratio of the metallocene catalyzed polypropylene to the Zeigler-Natta catalyzed polypropylene is from about 5:95 to 95:5, and in particular, from about 10:90 to 90:10, and more particularly, from about 20:80 to 80:20. In preferred embodiments, the ratio of metallocene catalyzed polypropylene to the Zeigler-Natta catalyzed polypropylene is from about 15:85, and in particular, from about 25:75, and more particularly, from about 30:70, and even more particularly to 35:65.

In certain embodiments, the first polypropylene polymer resin comprises a blend of a metallocene catalyzed polypropylene and a Zeigler-Natta catalyzed polypropylene in which the Zeigler-Natta polypropylene comprises at least 50 weight percent of the polymer blend, based on the total weight of the blend. For instance, the amount of the Zeigler-Natta catalyzed polypropylene in the blend is at least about 50 weight percent, at least 52 weight percent, at least 54 weight percent, at least 56 weight percent, at least 58 weight percent, at least 60 weight percent, at least 60 weight percent, at least 52 weight percent, at least 64 weight percent, at least 66 weight percent, at least 68 weight percent, 70 weight percent, at least 72 weight percent, at least 74 weight percent, at least 76 weight percent, at least 78 weight percent, at least 80 weight percent, at least 82 weight percent, and at least 84 weight percent, based on the total weight of the polymer blend.

### Elastomeric Polyolefin Polymer

Suitable elastomeric polyolefins may include polymers having polyethylene, polypropylene, polybutylene, and other olefinic polymers, and blends thereof provided they have elastomeric properties, such as extensibility, flexibility, and the like. The elastomeric polyolefin polymer may include both polyolefin homopolymers and polyolefin copolymers, and blends thereof.

In certain embodiments, the elastomeric polyolefin polymer is present as a minor component in the polypropylene blend. In certain other embodiments, the elastomeric polyolefin polymer is present as the major component in the polymer blend.

The amount of the elastomeric polyolefin in the polymer blend is typically from 1 to 99 weight percent, based on the total weight of the fiber, and in particular, from about 5 to 95 weight percent, and more particularly, 10 to 90 weight percent, and even more particularly, from about 15 to 85 weight percent, based on the total weight of the polymer blend.

In certain embodiments, the amount of the elastomeric polyolefin in the polymer blend is typically from 1 to 30 weight percent, based on the total weight of the fiber, and in particular, from about 5 to 20 weight percent, based on the total weight of the fiber. More particularly, the amount of the elastomeric polyolefin in the blend is from about 1 to 25 weight percent, based on the total weight of the blend. In particular, the amount of the elastomeric polyolefin may be from about 2 to 20 weight percent, such as from about 4 to 16 weight percent, from about 5 to 15 weight percent, and from about 6 to 14 weight percent, based on the total weight of the blend.

In certain embodiments, the elastomeric polyolefin is present in the polymer blend in an amount ranging from about 2 to 30 weight percent, and in particular, 5 to 25 weight percent, and more particularly, from about 8 to 20 weight percent, based on the total weight of the polymer blend.

In certain embodiments of the invention, the elastomeric polyolefin has a molecular weight that is less than the molecular weight of the first polypropylene resin. For example, the molecular weight of the elastomeric polyolefin may be from about 5 to 35 percent less than the molecular weight of the first polypropylene resin, such as from about 10 to 25 percent less, and in particular, from about 15 to 20 percent less.

Suitable examples of elastomeric polyolefins may include polymers in which propylene represents the majority component of the polymeric backbone, and as a result, any residual crystallinity possesses the characteristics of polypropylene crystals. Residual crystalline entities embedded in the propylene-based elastomeric molecular network may function as physical crosslinks, providing polymeric chain anchoring capabilities that improve the mechanical properties of the elastic network, such as high recovery, low set and low force relaxation.

Suitable examples of elastomeric polyolefins may include an elastic random poly(propylene/olefin) copolymer, an isotactic polypropylene containing stereoerrors, an isotactic/atactic polypropylene block copolymer, an isotactic polypropylene/random poly(propylene/olefin) copolymer block copolymer, a stereoblock elastomeric polyolefin, a syndiotactic polypropylene block poly(ethylene-co-propylene) block syndiotactic polypropylene triblock copolymer, an isotactic polypropylene block regioirregular polypropylene block isotactic polypropylene triblock copolymer, a polyethylene random (ethylene/olefin) copolymer block copolymer, a reactor blend polypropylene, a very low density polypropylene (or, equivalently, ultra low density polypropylene), a metallocene polypropylene, and combinations thereof.

In some embodiments, the elastomeric polyolefins include polypropylenes having both hard and soft segments in which the hard segments are of high crystallinity and the soft segments are amorphous or semi-amorphous. For example, suitable elastomeric polyolefin polymers including crystalline isotactic blocks and amorphous atactic blocks are described, for example, in U.S. Pat. Nos. 6,559,262, 6.518.378, and 6,169,151.

In certain embodiments, the elastomeric polyolefins include elastomeric random copolymers (RCPs) including propylene with a low level comonomer (e.g., ethylene or a higher α-olefin) incorporated into the backbone. For example, the elastomeric polyolefin may comprise a propylene copolymer comprising at least two different types of monomer units, one of which is propylene. Suitable examples of monomer units include, for example, ethylene and higher α-olefins in the range of C_{4 to} C₂₀, such as 1-butene, 4-methyl-1-pentene, 1-hexene, or 1-octene. And 1-decene, or mixtures thereof. Preferably, ethylene is copolymerized with propylene, so that the propylene copolymer comprises propylene units (polymer chain units derived from propylene monomers) and ethylene units (polymer chain units derived from ethylene monomers).

Typically, the units or comonomers of the propylene copolymer are derived from ethylene or at least one of C4-10 alpha-olefins are from 1% to 35%, or from 5% to about 35% by weight of the propylene-alpha-olefin copolymer. It may be present in an amount of wt %, or 7 wt % to 32 w1%, or 8 to about 25 wt %, or 8 w1% to 20 w1%, or even 8 w1% to 18 wt %. The comonomer content is such that the propylene-α-olefin copolymer preferably has an isothermal heat of fusion (“DSC”) of 75000 Gy (75 J/g) or less, a melting point of 100° C. or less, and a crystallinity of 2% to about 65%. In certain embodiments, the polypropylene copolymer includes tactic polypropylene and can preferably be adjusted to have a melt flow rate of 0.5 to 90 de/min.

In certain embodiments, the elastomeric polyolefin comprises a propylene-α-olefin copolymer having ethylene derived units. The propylene-α-olefin copolymer is 5% to 35%, or 5% to 20%, or 10% to 12%, or 15% to 20% by weight of the propylene-α-olefin copolymer. It may contain weight percent ethylene derived units. In some embodiments, the propylene-α-olefin copolymer consists essentially of units derived from propylene and ethylene, i.e, the propylene-α-olefin copolymer is ethylene and/or propylene used during polymerization.

In certain embodiments, the propylene-α-olefin copolymer may have a triad tacticity of three propylene units (measured by 13C NMR) of at least 75%, at least 80%, at least 82%, at least 85%, or at least 90% . . . “Triad tacticity” is determined as follows. The tacticity ratio (denoted herein as “m/r”) is determined by 13C nuclear magnetic resonance (“NMR”). The tacticity rate m/r N. Calculated by Cheng as defined in 17 MACROMOLECULES 1950 (1984), which is incorporated herein by reference. The notation “m” or “r” represents the stereochemistry of a pair of adjacent propylene groups, “m” refers to meso, and “r” refers to racemic. An m/r of 1.0 generally represents a syndiotactic polymer and an m/r ratio of 2.0 generally represents an atactic material. Isotactic materials theoretically have m/r ratios approaching infinity, and many byproduct atactic polymers have sufficient isotactic content to produce m/r ratios greater than 50.

Examples of suitable propylene-α-olefin copolymers may include VISTAMAXX® (ExxonMobil Chemical Company, Houston, Tex., USA), VERSIFY® (The Dow Chemical Company. Midland, Mich., USA).), Grades of TAFMER® XM or NOTIO® (Mitsui Company. Japan), and grades of SOFTEL® (Basell Polyfins of the Netherlands).

In certain embodiments, the elastomeric polyolefin comprises a low isotacticity homopolymer polypropylene (e.g., a polypropylene having an isotacticity [mmmm] from 30 to 70% by mol).

Accordingly, in certain embodiments the elastomeric polyolefin comprises a low isotacticity polypropylene that may be present in an amounts from about 1 to 30 weight percent, 2 to 245 weight percent, 3 to 22 weight percent, 4 to 21 weight percent, 5 to 20 weight percent, 6 to 19 weight percent, 7 to 18 weight percent, 8 to 17 weight percent, 9 to 16 weight percent, and 10 to 15 weight percent, based on the total weight of the polymer blend.

The low isotacticity polypropylene may generally be characterized by one or more of the following properties:
isotacticity: a meso pentad fraction [mmmm] of 20 to 70% by mol;
average number molecular weight (Mw) of 10,000 to 200,000;
a melting temperature from about 60 to 120° C.; and
a melt flow rate (MFR) greater than 40 g/10 min.

In addition to the above properties the low isotacticity polypropylene may have a B-viscosity from about 7,000 to 400,000 mPa, and a tensile modulus from about 80 to 120 MPa.

Low isotacticity polypropylenes polymers that are suitable generally have an isotacticity [mmmm] (% by mol) that is between about 20 and 70, and in particular, a [mmmm] between 30 and 60% by mol, and more particularly, a [mmmm] between 35 and 55% by mol. In one embodiment, the low isotacticity polypropylene has an isotacticity [mmmm] that is between about 40 and 50% by mol.

The stereochemistry (e.g., stereoregularity index ([mm]), meso pentad fraction [mmmm], the racemic pentad fraction [rrrr], the racemic-meso-racemic-meso pentad fraction [rmrm], and triad fractions [mm] [rr] and [mr]) of the low isotacticity polypropylene may be determined with an ¹³C-NMR spectrum according to the attribution of peaks proposed by A. Zambelli, et al., Macromolecules, No. 8, p. 687 (1975). A ¹³C-NMR, Model JNM-EX400, produced by JEOL Ltd. may be used to obtain the spectrum according to the following parameters:
Method: proton complete decoupling method;
Concentration: 220 mg/mL;
Solvent: mixed solvent of 1,2,4-trichlorobenzene and deuterated benzene (90/10 by volume);
Temperature: 130° C.;
Pulse width: 45°;
Pulse repetition time: 4 sec;
Accumulation: 10,000 times;

$$M = m / S \times 100 \qquad \text{<Calculation Expression>}$$

$$R = γ / S \times 100$$

$$S = P ββ + P αβ + P αγ$$

$$S = P ββ : 19.8 - 22.5 ppm$$

$$P αβ : 18. - 17.5 ppm$$

$$P αγ : 17.5 - 17.1 ppm$$

$$γ : racemic pentad chain : 20.7 - 20.3 ppm$$

$$m : meso pentad chain : 21.7 - 22.5 ppm .$$

In certain embodiments, the elastomeric polyolefin comprises a low isotactic polypropylene having an isotacticity [mmmm] (% by mol) that is greater than about 30, greater than about 31, greater than about 32, greater than about 33, greater than about 34, greater than about 35, greater than about 36, greater than about 37 greater than about 38, greater than about 39, greater than about 40, greater than about 41, greater than about 42, greater than about 43, greater than about 44, greater than about 45, greater than about 45, greater than about 47, greater than about 48, greater than about 49, greater than about 50, greater than about 51, greater than about 52, greater than about 53, greater than about 54, greater than about 55, greater than about 56, greater than about 57, greater than about 58, greater than about 59, and greater than about 60.

In one embodiment, the low isotacticity polypropylene has an isotacticity [mmmm] (% by mol) that is less than about 60, less than about 59, less than about 58, less than about 57, less than about 56, less than about 55, less than about 54, less than about 53, less than about 52, less than about 51, less than about 50, less than about 49, less than about 48, less than about 47, less than about 46, less than about 45, less than about 44, less than about 43, less than about 42, less than about 41, less than about 40, less than about 39, less than about 38, less than about 37, less than about 36, less than about 35, less than about 34, less than about 33, less than about 32, and less than about 31.

In some embodiments, the elastomeric polyolefin comprises a low isotacticity polypropylene may have a crystallinity that is from about 30 to 60 percent, such as between 35 and 55 percent, between 40, and 50 percent, and preferably, between 42 and 48 percent. In one embodiment, the low isotacticity polypropylene may have a crystallinity that is from about 44 to 46 percent. Crystallinity of the low isotacticity polypropylene may be measured in accordance with ASTM D-3418-15.

In one embodiment, the elastomeric polyolefin comprises a low isotacticity polypropylene typically having an MFR greater than 40 g/10 min and a molecular weight of less than 140,000 g/mol, and in particular, an MFR greater than 45 g/10 min and a molecular weight less than 134,200 g/mol. In a preferred embodiment, the elastomeric polyolefin comprises a low isotacticity polypropylene having a molecular weight between 124,200 g/mol and 134,200 g/mol and an MFR from about 45 to 55 g/10 min. Unless otherwise indicated MFR is measured in accordance with ASTM D-1238.

In certain embodiments, the elastomeric polyolefin comprises a low isotacticity polypropylene having a melting temperature that is greater than about 60° C., and in particular, from about 60 to 120° C., and more particularly, from about 60 to 100° C. In one embodiment, the low isotacticity polypropylene has a melting temperature that is from about 65 to 85° C., and in particular, from about 70 to 80° C. The melting temperature of low isotacticity polypropylene can be determined in accordance with ISO 306 Method A50.

In certain embodiments, the elastomeric polyolefin comprises a low tacticity polypropylene having a molecular weight ranging from about 30,000 to about 150,000 g/mol, and in particular, from about 44,200 to about 140,000 g/mol, and more particularly, from about 70,000 to 134,200 g/mol. In a preferred embodiment, the low isotacticity polypropylene has a molecular weight that is from about 128,000 to about 132,000 g/mol.

In one embodiment, the elastomeric polyolefin comprises a low isotacticity polypropylene may have a molecular weight less than one of the following: less than about 150,000 g/mol, less than about 144,200 g/mol, less than about 140,000 g/mol, less than about 138,000 g/mol, less than about 136,000 g/mol, less than about 134,000 g/mol, less than about 132,000 g/mol, less than about 130,000 g/mol, less than about 128,000 g/mol, less than about 126,000 g/mol, less than about 124,000 g/mol, less than about 122,000 g/mol, less than about 120,000 g/mol, less than about 118,000 g/mol, less than about 116,000 g/mol, less than about 114,000 g/mol, less than about 112,000 g/mol, less than about 110,000 g/mol, less than about 108,000 g/mol, less than about 106,000 g/mol, less than about 104,000 g/mol, less than about 102,000 g/mol, less than about 100,000 g/mol, less than about 98,000 g/mol, less than about 96,000 g/mol, less than about 94,000 g/mol, less than about 92,000 g/mol, less than about 90,000 g/mol, less than about 88,000 g/mol, less than about 86,000 g/mol, less than about 84,000 g/mol, less than about 82,000 g/mol, less than about 80,000 g/mol, less than about 78,000 g/mol, less than about 76,000 g/mol, less than about 74,000 g/mol, less than about 72,000 g/mol, or less than about 70,000 g/mol.

In some embodiments, the low isotacticity polypropylene has a molecular weight that is less than the molecular weight of the first polypropylene polymer resin in which it is blended. For example, in certain embodiments of the present invention, the percent difference in the molecular weight between the first polypropylene polymer and the low isotacticity polypropylene is from 5 to 150%. In one embodiment, the percent difference may be between 7 and 120%. In a preferred embodiment, the percent difference in the molecular weight between the first polypropylene polymer and the low isotacticity polypropylene is from about 20 to 35%, and more preferably, from about 25 to 30%.

In the context of the present invention, percent difference is calculated according to the following:

$$PERCENT DIFFERENCE = \frac{\vert {V}_{1} - {V}_{2} \vert}{[ \frac{( {V}_{1} + {V}_{2} )}{2} ]} \times 100$$

In one embodiment, the polypropylene polymer has a molecular weight of 172,000 g/mol and the low isotacticity weight polypropylene has a molecular weight that is about 130,000 to provide a percent difference of about 27.8%. In another embodiment, the first polypropylene may have a molecular weight of about 140,000 g/mol, and the low isotacticity polypropylene may have a molecular weight of 130,000 g/mol to provide a percent difference of about 7%. In a further embodiment, the first polypropylene may have a molecular weight of about 172,000 g/mol, and the low isotacticity polypropylene may have a molecular weight of 44,200 to provide a percent difference of about 117%

Examples of suitable low isotacticity polypropylenes are available from Idemitsu under the product name L-MODU™. Examples include S400 (˜2,600 MFR g/10 min, density of 0.87 g/cm³, and M_{w} 45 k g/mol); S600 (390 MFR g/10 min, density of 0.87 g/cm³, and M_{w} 75 k g/mol); and S901 (50 MFR g/10 min, density of 0.87 g/cm³, and M_{w} 130 k g/mol).

In other embodiments, the low isotacticity polypropylene may comprise a copolymer of ethylene and propylene units.

### Optional Components

In some embodiments, the fibers may include one or more additional additives that are blended with the polymer(s) during the melt extrusion phase. Examples of suitable additives include one or more of colorants, such as pigments (e.g., TiO₂), UV stabilizers, hydrophobic agents, hydrophilic agents, antistatic agent, elastomers, compatibilizers, antioxidants, anti-block agent, slip agents, surfactants, optical brighteners, flame retardants, antimicrobials, such as copper oxide and zinc oxide and the like.

In certain embodiments, the fibers of the nonwoven fabric have a monocomponent configuration.

In certain embodiments, the fibers of the nonwoven fabric have a multicomponent configuration, such as bicomponent configuration. For example, the fibers may have a sheath/core configuration, side-by-side-eccentric sheath/core configuration, islands in the sca configuration and the like.

In certain embodiments, the fibers comprise bicomponent fibers in which the fibers have a first polymer component and a second polymer component in which each component comprises a distinct region of the fiber. In such embodiments, the elastomeric polyolefin may be present in the first polymer component, the second polymer component, or may be present in both the first and second polymer components.

In certain embodiments, the configuration of multicomponent fibers is a side-by-side arrangement wherein a first polymer component defines a first continuous distinct zone extending along the length of the fiber, and a second polymer component defines a second continuous distinct zone extending along the length of the fiber. Both the first and second polymer components define at least a portion of the outer surface of the continuous fibers. In certain embodiments, the first and second distinct zones of the side-by-side continuous fibers are present in ratios ranging from 10:90 to 90:10, and in particular, from about 40:60 to 60:40, and more particularly, from about 50:50. Side-by-side configurations are particularly useful in the preparation of crimped fibers. Other configurations that may be useful in the preparation of crimped fibers include eccentric sheath/core and D-centric sheath/core configurations.

A preferred configuration is a sheath/core arrangement wherein a first component, the sheath, substantially surrounds a second component, the core. The resulting sheath/core bicomponent fiber may have a round or non-round cross-section. Other structured fiber configurations as known in the art can be used including, segmented pie, islands-in-the-sea and tipped multilobal structures.

In certain embodiments, the fibers are bicomponent in which a first polymer component defines a sheath of the fiber, and a second polymer component defines a core of the fiber. Generally, the weight percentage of the sheath to that of the core in the fibers may vary widely depending upon the desired properties of the nonwoven fabric. For example the weight ratio of the sheath to the core may vary between about 5:95 to 95:5, such as from about 10:90 to 90:10, and in particular from about 20:80 to 80:20. In a preferred embodiment, the weight ratio of the sheath to the core is about 25:75 to 35:65, with a weight ratio of about 30:70 to 50:50 being preferred.

In certain embodiments, the fibers have a bicomponent structure in which the core and sheath both comprise the same type of first polypropylene polymer, and only the sheath or the core includes the elastomeric polyolefin that is present in an amount that is from about 1 to 99 weight percent, based on the total weight of the component in which the elastomeric polyolefin is present, and in particular, from about 5 to 30 weight percent, and more particularly from about 10 to 25 weight percent, and even more particularly, from about 15 to 20 weight percent, based on the total weight of the sheath component.

In certain embodiments, the elastomeric polyolefin may be present in only the core and not the sheath of a bicomponent fiber, only present in the sheath and not the core, or the high elastomeric polyolefin may be present in both the sheath and the core. In other embodiments, the elastomeric polyolefin may be present in a polymer blend in both the first and second components, but not at the same concentration. For example, the amount of the elastomeric polyolefin in the polymer blend of the sheath may be more or less than the amount of the elastomeric polyolefin in the polymer blend of the core.

In certain embodiments, the core comprises a polymer blend comprising the first polypropylene polymer resin and the elastomeric polyolefin while the sheath comprises a different polymer or polymer blend than that of the core. In such embodiments, the sheath may comprise a wide variety of polymers may be used in the preparation of nonwoven fabrics in accordance with embodiments of the present disclosure.

In such embodiments, the first polymer component, such as the sheath, may comprise a wide variety of different polymers and polymeric blends.

Examples of suitable polymers for preparing such multicomponent fibers include polyolefins, such as polypropylene and polyethylene, and copolymers thereof, polyesters, such as polyethylene terephthalate (PET), polytrimethylene terephthalate (PTT), and polybutylene terephthalate (PBT), nylons, polystyrenes, polyurethanes, copolymers, and blends thereof, and other synthetic polymers that may be used in the preparation of fibers. In some embodiment, the polymer can be selected from the group consisting of: polyolefins, polyesters, polyethylene terephthalates, polybutylene terephthalates, polycyclohexylene dimethylene terephthalates, polytrimethylene terephthalates, polymethyl methacrylates, polyamides, nylons, polyacrylics, polystyrenes, polyvinyls, polytetrafluoroethylenes, ultrahigh molecular weight polyethylenes, very high molecular weight polyethylenes, high molecular weight polyethylenes, polyether ether ketones, non-fibrous plasticized celluloses, polyethylenes, polypropylenes, polybutylenes, polymethylpentenes, low-density polyethylenes, linear low-density polyethylenes, high-density polyethylenes, polystyrenes, acrylonitrile-butadiene-styrenes, styrene-acrylonitriles, styrene triblock and styrene tetra block copolymers, styrene-butadienes, styrene-maleic anhydrides, ethylene vinyl acetates, ethylene vinyl alcohols, polyvinyl chlorides, cellulose acetates, cellulose acetate butyrates, plasticized cellulosics, cellulose propionates, ethyl cellulose, natural fibers, any derivative thereof, any polymer blend thereof, any copolymer thereof or any combination thereof.

In further embodiments, nonwoven fabrics nonwoven fabrics in accordance with one or more embodiments of the invention may be prepared comprising multicomponent fibers in which the fibers comprises a first polymer component comprising the polymer blend of the first polypropylene polymer resin and the elastomeric polypropylene, and a second polymer component comprising a bio-based material, and in particular, a bio-based polymers. In contrast to polymers derived from petroleum sources, bio-based polymers are generally derived from a bio-based material. In some embodiments, a bio-based polymer may also be considered biodegradeable. A special class of biodegradable product made with a bio-based material might be considered as compostable if it can be degraded in a composting environment. The European standard EN 13432. “Proof of Compostability of Plastic Products” may be used to determine if a fabric or film comprised of sustainable content could be classified as compostable.

In certain embodiments, the nonwoven fabric may comprise fibers comprising a bio-based polymer and the polymer blend as described above. In some embodiments, the fibers may have a bicomponent configuration in which the first polymer component comprises the polymer blend comprising the first polypropylene and the elastomeric polyolefin and a second polymer component comprising a bio-based polymer, such as a sheath/core configuration in which the polymer blend comprising the first polypropylene and the elastomeric polyolefin comprises the sheath and the bio-based polymer comprises the core of the fiber.

In one embodiment, bio-based polymers for use may include aliphatic polyester based polymers, such as polylactic acid, and bio-based derived polyethylene.

Aliphatic polyesters useful in the present invention may include homo- and copolymers of poly(hydroxyalkanoates), and homo- and copolymers of those aliphatic polyesters derived from the reaction product of one or more polyols with one or more polycarboxylic acids that are typically formed from the reaction product of one or more alkanediols with one or more alkanedicarboxylic acids (or acyl derivatives). Polyesters may further be derived from multifunctional polyols, e.g. glycerin, sorbitol, pentaerythritol, and combinations thereof, to form branched, star, and graft homo- and copolymers. Polyhydroxyalkanoates generally are formed from hydroxyacid monomeric units or derivatives thereof. These include, for example, polylactic acid, polyhydroxybutyrate, polyhydroxyvalerate, polycaprolactone and the like. Miscible and immiscible blends of aliphatic polyesters with one or more additional semicrystalline or amorphous polymers may also be used.

One useful class of aliphatic polyesters are poly(hydroxyalkanoates), derived by condensation or ring-opening polymerization of hydroxy acids, or derivatives thereof. Suitable poly(hydroxyalkanoates) may be represented by the formula: H(O—R—C(O)—)ₙOH where R is an alkylene moiety that may be linear or branched having 1 to 20 carbon atoms, preferably 1 to 12 carbon atoms optionally substituted by catenary (bonded to carbon atoms in a carbon chain) oxygen atoms; n is a number such that the ester is polymeric, and is preferably a number such that the molecular weight of the aliphatic polyester is at least 10,000, preferably at least 30,000, and most preferably at least 50,000 daltons. In certain embodiments, the molecular weight of the aliphatic polyester is typically less than 1,000,000, preferably less than 500,000, and most preferably less than 300,000 daltons. R may further comprise one or more caternary (i.e. in chain) ether oxygen atoms. Generally, the R group of the hydroxy acid is such that the pendant hydroxyl group is a primary or secondary hydroxyl group.

Useful poly(hydroxyalkanoates) include, for example, homo- and copolymers of poly(3-hydroxybutyrate), poly(4-hydroxybutyrate), poly(3-hydroxyvalerate), poly(lactic acid) (as known as polylactide), poly(3-hydroxypropanoate), poly(4-hydropentanoate), poly(3-hydroxypentanoate), poly(3-hydroxyhexanoate), poly(3-hydroxyheptanoate), poly(3-hydroxyoctanoate), polydioxanone, polycaprolactone, and polyglycolic acid (i.e. polyglycolide). Copolymers of two or more of the above hydroxy acids may also be used, for example, poly(3-hydroxybutyrate-co-3-hydroxyvalerate), poly(lactate-co-3-hydroxypropanoate), poly(glycolide-co-p-dioxanone), and poly(lactic acid-co-glycolic acid). Blends of two or more of the poly(hydroxyalkanoates) may also be used, as well as blends with one or more semicrystalline or amorphous polymers and/or copolymers.

The aliphatic polyester may be a block copolymer of poly(lactic acid-co-glycolic acid). Aliphatic polyesters useful in the inventive compositions may include homopolymers, random copolymers, block copolymers, star-branched random copolymers, star-branched block copolymers, dendritic copolymers, hyperbranched copolymers, graft copolymers, and combinations thereof.

Another useful class of aliphatic polyesters includes those aliphatic polyesters derived from the reaction product of one or more alkanediols with one or more alkanedicarboxylic acids (or acyl derivatives). Such polyesters have the general formula:

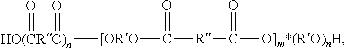

where R′ and R″ each represent an alkylene moiety that may be linear or branched having from 1 to 20 carbon atoms, preferably 1 to 12 carbon atoms, and m is a number such that the ester is polymeric, and is preferably a number such that the molecular weight of the aliphatic polyester is at least 10,000, preferably at least 30,000, and most preferably at least 50,000 daltons, but less than 1,000,000, preferably less than 500,000 and most preferably less than 300,000 daltons. Each n is independently 0 or 1. R′ and R″ may further comprise one or more caternary (i.e. in chain) ether oxygen atoms.

Examples of aliphatic polyesters include those homo- and copolymers derived from (a) one or more of the following diacids (or derivative thereof): succinic acid; adipic acid; 1,12 dicarboxydodecane; fumaric acid; glutartic acid; diglycolic acid; and maleic acid; and (b) one of more of the following diols: ethylene glycol; polyethylene glycol; 1,2-propane diol; 1,3-propanediol; 1,2-propanediol; 1,2-butanediol; 1,3-butanediol; 1,4-butanediol; 2,3-butanediol; 1,6-hexanediol; 1,2 alkane diols having 5 to 12 carbon atoms; diethylene glycol; polyethylene glycols having a molecular weight of 300 to 10,000 daltons, and preferably 400 to 8,000 daltons; propylene glycols having a molecular weight of 300 to 4000 daltons; block or random copolymers derived from ethylene oxide, propylene oxide, or butylene oxide; dipropylene glycol; and polypropylene glycol, and (c) optionally a small amount, i.e., 0.5-7.0 mole percent of a polyol with a functionality greater than two, such as glycerol, neopentyl glycol, and pentacrythritol.

Such polymers may include polybutylene succinate homopolymer, polybutylene adipate homopolymer, polybutyleneadipate-succinate copolymer, polyethylenesuccinate-adipate copolymer, polyethylene glycol succinate homopolymer and polyethylene adipate homopolymer.

Commercially available aliphatic polyesters include poly(lactide), poly(glycolide), poly(lactide-co-glycolide), poly(L-lactide-co-trimethylene carbonate), poly(dioxanone), poly(butylene succinate), and poly(butylene adipate).

The term “aliphatic polyester” covers—besides polyesters which are made from aliphatic and/or cycloaliphatic components exclusively also polyesters which contain besides aliphatic and/or cycloaliphatic units, aromatic units, as long as the polyester has substantial bio-based content.

In addition to PLA based resins, nonwoven fabrics in accordance with embodiments of the invention may include other polymers derived from an aliphatic component possessing one carboxylic acid group and one hydroxyl group, which are alternatively called polyhydroxyalkanoates (PHA). Examples thereof are polyhydroxybutyrate (PHB), poly-(hydroxybutyrate-co-hydroxyvaleterate) (PHBV), poly-(hydroxybutyrate-co-polyhydroxyhexanoate) (PHBH), polyglycolic acid (PGA), poly-(epsilon-caprolactione) (PCL) and preferably polylactic acid (PLA).

Examples of additional polymers that may be used in embodiments of the invention include polymers derived from a combination of an aliphatic component possessing two carboxylic acid groups with an aliphatic component possessing two hydroxyl groups, and are polyesters derived from aliphatic diols and from aliphatic dicarboxylic acids, such as polybutylene succinate (PBS), polyethylene succinate (PES), polybutylene adipate (PBA), polyethylene adipate (PEA), polytetramethy-lene adipate/terephthalate (PTMAT).

Useful aliphatic polyesters include those derived from semicrystalline polylactic acid. Poly(lactic acid) or polylactide (PLA) has lactic acid as its principle degradation product, which is commonly found in nature, is non-toxic and is widely used in the food, pharmaceutical and medical industries. The polymer may be prepared by ring-opening polymerization of the lactic acid dimer, lactide. Lactic acid is optically active and the dimer appears in four different forms: L,L-lactide, D,D-lactide, D,L-lactide (meso lactide) and a racemic mixture of L,L- and D,D-. By polymerizing these lactides as pure compounds or as blends, poly(lactide) polymers may be obtained having different stereochemistries and different physical properties, including crystallinity. The L,L- or D,D-lactide yields semicrystalline poly(lactide), while the poly(lactide) derived from the D,L-lactide is amorphous.

Generally, polylactic acid based polymers are prepared from dextrose, a source of sugar, derived from field corn. In North America corn is used since it is the most economical source of plant starch for ultimate conversion to sugar. However, it should be recognized that dextrose can be derived from sources other than corn. Sugar is converted to lactic acid or a lactic acid derivative via fermentation through the use of microorganisms. Lactic acid may then be polymerized to form PLA. In addition to corn, other agriculturally-based sugar sources may be used including rice, sugar beets, sugar cane, wheat, cellulosic materials, such as xylose recovered from wood pulping, and the like.

The polylactide preferably has a high enantiomeric ratio to maximize the intrinsic crystallinity of the polymer. The degree of crystallinity of a poly(lactic acid) is based on the regularity of the polymer backbone and the ability to crystallize with other polymer chains. If relatively small amounts of one enantiomer (such as D-) is copolymerized with the opposite enantiomer (such as L-) the polymer chain becomes irregularly shaped, and becomes less crystalline. For these reasons, when crystallinity is favored, it is desirable to have a poly(lactic acid) that is at least 85% of one isomer, at least 90% of one isomer, or at least 95% of one isomer in order to maximize the crystallinity.

In some embodiments, an approximately equimolar blend of D-polylactide and L-polylactide is also useful. In certain embodiments, this blend forms a unique crystal structure having a higher melting point than does either the D-poly(lactide) and L-(polylactide) alone, and has improved thermal stability.

Copolymers, including block and random copolymers, of poly(lactic acid) with other aliphatic polyesters may also be used. Useful co-monomers include glycolide, beta-propiolactone, tetramethylglycolide, beta-butyrolactone, gamma-butyrolactone, pivalolactone, 2-hydroxybutyric acid, alpha-hydroxyisobutyric acid, alpha-hydroxyvaleric acid, alpha-hydroxyisovaleric acid, alpha-hydroxycaproic acid, alpha-hydroxycthylbutyric acid, alpha-hydroxyisocaproic acid, alpha-hydroxy-beta-methylvaleric acid, alpha-hydroxyoctanoic acid, alpha-hydroxydecanoic acid, alpha-hydroxymyristic acid, and alpha-hydroxystearic acid.

Blends of poly(lactic acid) and one or more other aliphatic polyesters, or one or more other polymers may also be used. Examples of useful blends include poly(lactic acid) and poly(vinyl alcohol), polyethylene glycol/polysuccinate, polyethylene oxide, polycaprolactone and polyglycolide.

In certain preferred embodiments, the aliphatic polyester component comprises a PLA based resin. A wide variety of different PLA resins may be used to prepare nonwoven fabrics in accordance with embodiments of the invention. The PLA resin should have proper molecular properties to be spun in spunbond processes. Examples of suitable include PLA resins are supplied from NatureWorks LLC, of Minnetonka, Minn. 55345 such as, grade 6752D, 6100D, and 6202D, which are believed to be produced as generally following the teaching of U.S. Pat. Nos. 5,525,706 and 6,807,973 both to Gruber et al. Other examples of suitable PLA resins may include L130, L175, and LX175, all from Corbion of Arkelsedijk 46, 4206 A C Gorinchem, the Netherlands.

In some embodiments, the inventive nonwoven fabrics may comprise bio-based polymer components of biodegradable products that are derived from an aliphatic component possessing one carboxylic acid group (or a polyester forming derivative thereof, such as an ester group) and one hydroxyl group (or a polyester forming derivative thereof, such as an ether group) or may be derived from a combination of an aliphatic component possessing two carboxylic acid groups (or a polyester forming derivative thereof, such as an ester group) with an aliphatic component possessing two hydroxyl groups (or a polyester forming derivative thereof, such as an ether group).

Additional nonlimiting examples of bio-based polymers include polymers directly produced from organisms, such as polyhydroxyalkanoates (e.g., poly(beta-hydroxyalkanoate), poly(3-hydroxybutyrate-co-3-hydroxyvalerate, NODAX™), and bacterial cellulose; polymers extracted from plants and biomass, such as polysaccharides and derivatives thereof (e.g., gums, cellulose, cellulose esters, chitin, chitosan, starch, chemically modified starch), proteins (e.g., zein, whey, gluten, collagen), lipids, lignins, and natural rubber; and current polymers derived from naturally sourced monomers and derivatives, such as bio-polyethylene, bio-polypropylene, polytrimethylene terephthalate, polylactic acid, NYLON 11, alkyd resins, succinic acid-based polyesters, and bio-polyethylene terephthalate.

In some embodiments, the bio-based polymer may comprise bio-based polyethylene, bio-based polypropylene, and bio-based polyesters, such as bio-based PET, that are derived from a biological source. For example, bio-based polyethylene can be prepared from sugars that are fermented to produce ethanol, which in turn is dehydrated to provide ethylene. An example of a suitable sugar cane derived polyethylene is available from Braskem S.A. under the product name PE SHA7260.

### Fabric Properties

In accordance with certain embodiments, for example, the nonwoven fabric may have a basis weight from about 5 grams per square meter (gsm) to about 150 gsm, depending on the number of layers in the fabric and the composition of each layer.

In particular, the fabric may have a basis weight from about 8 gsm to about 150 gsm. In certain embodiments, for example, the fabric may comprise a basis weight from about 10 gsm to about 70 gsm. In further embodiments, for instance, the fabric may have a basis weight from about 11 gsm to about 40 gsm. In one embodiment, the fabric may have a basis weight from about 15 gsm to about 25 gsm. As such, in certain embodiments, the fabric may have a basis weight from at least about any of the following: 5, 6, 7, 8, 9, 10, 11, 12, 12, 14, and 15 gsm and/or at most about 150, 100, 70, 60, 50, 40, and 30 gsm (e.g., about 9-60 gsm, about 11-40 gsm, about 20 to 35 gsm, etc.).

Advantageously, nonwoven fabrics in accordance with embodiments of the disclosure may have fibers typically have a linear mass density from about 0.7 dtex to about. 1.7 dtex. In certain embodiments, the fibers have a linear mass density from at least about any of the following: 0.81, 0.85, 0.90, 0.95, 1.0, 1.05, 1.10, 1.15, 1.20, 1.25, 1.30, 1.35, 1.40, 1.45, 1.50, 1.55, 1.60, and 1.65, and/or at most about 1.65, 1.60, 1.55, 1.50, 1.45, 1.40, 1.35, 1.30, 1.25, 1.20, 1.15, 1.10, 1.05, 1.00, 0.95, 0.90, 0.85, and 0.80 dtex (e.g., about 0.70-1.65 dtex, about 0.75-1.60 dtex, 0.8-1.4, etc.).

In certain embodiments, nonwoven fabrics in accordance with embodiments of the invention exhibit improvements in one or more of elongations, abrasion resistance, fiber fineness, and softness in comparison to a similarly prepared nonwoven fabric with the exception that the similarly fabric does not include the elastomeric polyolefin in the polymer blend and the filaments were attenuated and drawn at an operating cabin pressure of less than 4,200 Pa

In certain embodiments, the similarly prepared fabric is substantially identical (for example polymer chemistry with the exception of the elastomeric polyolefin and operating cabin pressure) to the inventive fabric. Some variations in process conditions used in the similarly prepared nonwoven fabric may exist, such as, for example, slight variations in calender temperatures and pressures.

In certain embodiments of the disclosure, nonwoven fabrics in accordance with aspects of the disclosure are characterized by one or more of a decrease in fiber fineness (dtex) of at least 5%, at least 10%, at least 15%, at least 20%, at least 25%, at least 30%, and at least 35% in comparison to an identically prepared nonwoven fabric that does not include the elastomeric polyolefin blended with the polypropylene resin and in which the filaments were drawn and attenuated at a cabin pressure of less than 4,200 Pa.

In certain preferred embodiments, nonwoven fabrics in accordance with aspects of the disclosure are characterized by having an average fiber fineness ranging from about 0.8 to 1.6 dtex, and in particular, from about 0.9 to 1.4. and more particularly, from about 1.2 to 1.35 dtex.

In certain embodiments, the nonwoven fabric exhibits a percent decrease in fiber dtex from about 5 to 60%, such as a percent decrease of 15 to 50%, a percent decrease of 10 to 30%, and a percent decrease of 15 to 25% in comparison to an identically prepared fabric with the exception that the identically prepared fabric does not include the elastomeric polyolefin and in which the filaments were drawn and attenuated at a cabin pressure of less than 4,200 Pa.

In addition, nonwoven fabrics in accordance with embodiments of the invention demonstrate improvements in softness as demonstrated by both MD and CD bending.

In certain embodiments, the nonwoven fabric exhibits a machine direction bending softness ranging from 30 to 50 mm, and a cross direction bending softness ranging from 15 to 40 mm.

In certain embodiments, the nonwoven fabric exhibits a machine direction softness of less than one or more of 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, and 20 mm.

In certain embodiments, the nonwoven fabric exhibits a machine direction softness of at least one or more of 30 mm, 32 mm, 34 mm, 36 mm, 38 mm, and 40 mm, 42 mm, 44 mm, 46 mm, 48 mm, and 50 mm, and a cross direction softness of at least one or more of 16 mm, 18 mm, 20 mm, least 24 mm, 26 mm, 28 mm, 30 mm, 32 mm, 34 mm, 36 mm, 38 mm, and 40 mm, 42 mm, 44 mm, 46 mm, 48 mm, and 50 mm.

In certain embodiments, the nonwoven fabric has an average machine/cross direction bending softness ranging from 25 to 40 mm, and in particular, from about 28 to 38 mm. For example, the nonwoven fabric may have an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.

In some embodiments, the exhibits a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric exhibits an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and is drawn and attenuated at a cabin pressure of less than 4,200 Pa.

In some embodiments, the nonwoven fabrics exhibit an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the filaments were drawn and attenuated at a cabin pressure of less than 4,200 Pa.

In some embodiments, the nonwoven fabrics exhibit an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the filaments were drawn and attenuated at a cabin pressure of less than 4,200 Pa.

Advantageously, the improvements in softness and fiber fineness are accompanied by minor to no reduction in mechanical properties in comparison to an identically prepared fabric with the exception that the identically prepared fabric does not include the elastomeric polyolefin and in which the filaments were drawn and attenuated at a cabin pressure of less than 4,200 Pa. In some embodiments, the inventive nonwoven fabrics have exhibited improvements in both tensile strengths and elongations.

For example, nonwoven fabrics may exhibit an increase in MD and CD tensile strengths ranging from about 2 to 15% in comparison to an identically prepared fabric with the exception that the identically prepared fabric does not include the elastomeric polyolefin and in which the filaments were drawn and attenuated at a cabin pressure of less than 4,200 Pa.

In certain embodiments, nonwoven fabrics may exhibit an increase in percent elongations in one or more of machine or cross direction ranging from about 5 to 30% in comparison to an identically prepared fabric with the exception that the identically prepared fabric does not include the elastomeric polyolefin and in which the filaments were drawn and attenuated at a cabin pressure of less than 4,200 Pa.

In certain embodiments, nonwoven fabrics in accordance with embodiments of the invention may be characterized by having one or more of the following characteristics:
a) an average fiber fineness ranging from about 0.8 to 1.6 dtex, and in particular, from about 0.9 to 1.4. and more particularly, from about 1.2 to 1.35 dtex;
b) the nonwoven fabric exhibits a percent decrease in fiber dtex from about 5 to 60%, such as a percent decrease of 15 to 50%, in comparison to an identically prepared fabric with the exception that the identically prepared fabric does not include the elastomeric polyolefin and in which the filaments were drawn and attenuated at a cabin pressure of less than 4,200 Pa;
c) the nonwoven fabric exhibits a machine direction bending softness ranging from 30 to 50 mm and a cross direction bending softness ranging from 15 to 40 mm;
d) the nonwoven fabric exhibits a machine direction softness of less than one or more of 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, 20 mm, 18 mm, and 16 mm;
e) a machine direction softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, 44 mm, 46 mm, 48 mm, and 50 mm;
f) an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.
g) a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the filaments were drawn and attenuated at a cabin pressure of less than 4,200 Pa;
h) an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the filaments were drawn and attenuated at a cabin pressure of less than 4,200 Pa;
i) an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the filaments were drawn and attenuated at a cabin pressure of less than 4,200 Pa;
j) an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the filaments were drawn and attenuated at a cabin pressure of less than 4,200 Pa;
k) an increase in MD and CD tensile strengths ranging from about 2 to 15% in comparison to an identically prepared fabric with the exception that the identically prepared fabric does not include the elastomeric polyolefin and in which the filaments were drawn and attenuated at a cabin pressure of less than 4,200 Pa; and
l) an increase in percent elongations in one or more of machine or cross direction ranging from about 5 to 30% in comparison to an identically prepared fabric with the exception that the identically prepared fabric does not include the elastomeric polyolefin and in which the filaments were drawn and attenuated at a cabin pressure of less than 4,200 Pa.

In certain embodiments, nonwoven fabrics in accordance with embodiments of the invention have at two or more of the following characteristics:
a) an average fiber fineness ranging from about 0.8 to 1.6 dtex, and in particular, from about 0.9 to 1.4. and more particularly, from about 1.2 to 1.35 dtex;
b) a percent decrease in fiber dtex from about 5 to 60%, such as a percent decrease of one or more of 10 to 50% or 15 to 25%, in comparison to an identically prepared fabric with the exception that the identically prepared fabric does not include the elastomeric polyolefin and in which the filaments were drawn and attenuated at a cabin pressure of less than 4,200 Pa;
c) a machine direction bending softness ranging from 30 to 50 mm and a cross direction bending softness ranging from 15 to 40 mm;
d) a machine direction softness of less than one or more of 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, 20 mm¹⁸ mm, and 16 mm;
e) a machine direction softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, 44 mm, 46 mm, 48 mm, and 50 mm;
f) an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.
g) a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the filaments were drawn and attenuated at a cabin pressure of less than 4,200 Pa;
h) an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the filaments were drawn and attenuated at a cabin pressure of less than 4,200 Pa;
i) an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the filaments were drawn and attenuated at a cabin pressure of less than 4,200 Pa;
j) an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the filaments were drawn and attenuated at a cabin pressure of less than 4,200 Pa;
k) an increase in MD and CD tensile strengths ranging from about 2 to 15% in comparison to an identically prepared fabric with the exception that the identically prepared fabric does not include the elastomeric polyolefin and in which the filaments were drawn and attenuated at a cabin pressure of less than 4,200 Pa; and
l) an increase in percent elongations in one or more of machine or cross direction ranging from about 5 to 30% in comparison to an identically prepared fabric with the exception that the identically prepared fabric does not include the elastomeric polyolefin and in which the filaments were drawn and attenuated at a cabin pressure of less than 4,200 Pa.

In certain embodiments, nonwoven fabrics in accordance with embodiments of the invention may be characterized by having at least the following characteristics:
a) an average fiber fineness ranging from about 0.8 to 1.6 dtex, and in particular, from about 0.9 to 1.4. and more particularly, from about 1.2 to 1.35 dtex;
b) percent decrease in fiber dtex from about 5 to 30%, such as a percent decrease of 15 to 25%, in comparison to an identically prepared fabric with the exception that the identically prepared fabric does not include the elastomeric polyolefin and in which the filaments were drawn and attenuated at a cabin pressure of less than 4,200 Pa; and
c) a machine direction bending softness ranging from 30 to 50 mm and a cross direction bending softness ranging from 15 to 40 mm.

Softness Bending is measured in accordance with WSP90.1.

In certain embodiments, nonwoven fabrics in accordance with embodiments of the invention may be characterized by having a surface with a bonding pattern having a percent bonded area from about 9.6 to 14%, an average individual bond surface area from about 0.10 to 0.25 mm², an average bond point packing value from about 4 to 7.25 mm⁻¹, and one or more of the preceding properties a)-l).

### System and Method for Preparing the Nonwoven Fabric

Certain aspects of the invention provide systems and methods for preparing a nonwoven fabric in accordance with embodiments described previously discussed.

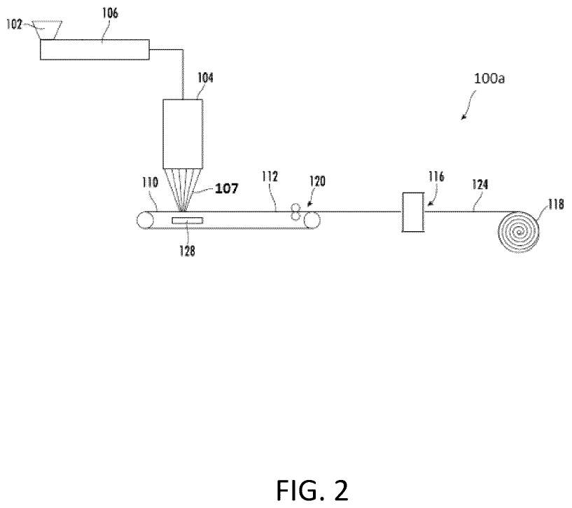

With reference to FIG. 2, for example, a schematic diagram of a spunbond nonwoven fabric preparation system in accordance with certain embodiments of the invention is illustrated and broadly designated by reference character 100a. As shown in FIG. 2, a first polymer source (i.e. hopper) 102 is in fluid communication with a spunbond spin beam 104 via the extruder 106. It should be understood that other nonwoven forming systems may be used in accordance with certain embodiments of the invention, such as meltblown, carded, air bonded, resin bonded, spunlace, and the like.

In certain embodiments, a source of elastomeric polyolefin polymer (not shown) is in fluid communication with either the hopper 102 or the extruder 106. The elastomeric polyolefin may be blended with the polypropylene polymer in the hopper or may be separately metered into the extruder.

In certain embodiments, the first polymer source may provide a stream of a molten or semi-molten polymer resin. Following extrusion, the extruded polymer stream containing the blend of the polymer and elastomeric polyolefin are introduced into the spunbond spin beam 104 at which point a plurality of streams of molten or semi-molten polymer are introduced into a die block (not shown) of a spunbond spin beam. The die block includes a plurality of multi-rows of fluid orifices that extend laterally in the cross direction of the spunbond spinbeam. As the extruded filaments exit the fluid orifices, cooling air is directed laterally against the extruded filaments, and then flows downwardly with the filaments through a partially enclosed cabin system (not shown), which cools, draws, and attenuates the filaments. Conventionally, the throughput of the polymer streams and air pressure (also referred to as cabin pressure) within the cabin system are maintained at a cabin pressure of less than 4,000 Pa to avoid undesirous deformation of the fibers and/or fiber breakage that may occur as the filaments are drawn through the cabin system.

In certain embodiments of the present disclosure, the inventors have discovered that the inclusion of the elastomeric polyolefin in the polymer blend allows the use of a higher cabin pressure without causing fiber breakage or fiber deformation. In turn, the high cabin pressure results in the formation of fine fibers having a decrease in denier than would normally be possible without the inclusion of the elastomeric polymer.

In conventional spunbond processes for the formation of polypropylene fibers, the system and process is normally operated at cabin pressures of less than 4,000 Pa. In embodiments of the present disclosure, the presence of the elastomeric polyolefin permits higher cabin pressures without experiencing fiber breakage and/or fiber deformation. Fiber breakage is particularly undesirable because it results in an unsightly and/or defective material, and may require the spunbond process to be stopped, cleaned, and restarted.

In certain embodiment, the polypropylene fibers are spun at a cabin pressures ranging in excess of 4,200 Pa, and in particular, from about 4,200 to 9,500 Pa.

While not wishing to be bound by theory, it is believed that the presence of the elastomeric polyolefin in the polymer blend reduces the maximum strain rate and stretching stress experienced by the fibers, which allows them to be subjected to higher cabin pressures without fiber breakage or deformation. The higher cabin pressures results in additional extension and elongation of the fibers as they are drawn and attenuated within the cabin system. As a result, fine fibers of lower denier are obtained, which is not possible with an identical process that does not include the elastomeric polyolefin in the polymer blend.

After exiting the cabin system, the spunbond fibers 107 are deposited on the collection surface 110 to produce a web of filaments. At this stage, the filaments may comprise a web 112 of filaments that are unbonded or slightly bonded to each other.

In certain embodiments, an optional bonding unit 116 is disposed downstream of the collection surface 110 and is configured and arranged to thermally bond fibers to each other to form a coherent web. During thermal bonding the web 112 of fibers, the fibers are heated to a temperature that is sufficient to soften at least one polymer component comprising fibers of web 112 to produce a bonded nonwoven fabric 124. In certain embodiments, the bonded nonwoven fabric 124 moves to a winder 118, where the fabric is then wound onto rolls.

In certain embodiments, the bonding unit comprises an air through bonder in which the fibers are exposed to one or more streams of heated gas, such as air. In other embodiments, the bonding unit may comprise a calender bonding unit comprising a pair of cooperating heated rolls in which at least one of the rolls includes a plurality of raised bonding points on a surface thereof. The bonding points can be used to impart a bonding pattern on at least one surface of the nonwoven fabric. In some embodiments, the calender comprises a pair of cooperating rolls in which a first roll comprises an engraved patterned roll having a plurality of bonding points extending from a surface thereof, and the second roll comprises smooth or anvil surface. During bonding, the web of spunbond fibers passes between the pair cooperating rolls, which are heated to a temperature that is sufficient to soften at least one polymer component comprising filaments of web such that the softened polymer component fuses and bonds to adjacent filaments within the web to produce a bonded nonwoven fabric.

The bonding points of the engraved roll may have a variety of different shapes, such as rods that extend in the cross direction, machine direction, or both, oval elliptical, square, diamond, hexagonal, circular, or the like. The surface area and density of the bonding points may be selected so that 5 to 30 percent of the surface area of the nonwoven fabric is thermally bonding with individual spaced apart thermal bonds. In certain embodiments, from about 5 to 20 percent, and in particular, from about 8 to 14 percent, of the surface area of the nonwoven fabric is thermally bonded.

In certain embodiments, it has also been discovered that improvements in abrasion resistance and softness of the nonwoven fabric can be obtained with a calender bonding unit in which the engraved patterned roll is configured with bonding points to provide a first bond pattern on the surface of the nonwoven fabric comprising a plurality of alternating arrays composed of individual bond points extending in the machine direction, cross direction, diagonal direction of the nonwoven fabric. In the first bond pattern, an overall percentage of bonded surface area of the nonwoven fabric is less than 14%, the surface area of the individual bond points is from about 0.10 to 0.60 square millimeters (mm²), the bond point packing value is greater than about 3.5 mm⁻¹, and the bond density is from about 20 to 60 individual point bonds per square centimeter (cm²). More specifically, it has been observed that bonding patterns meeting these features helps to provide a nonwoven fabric having improved softness and abrasion resistance in comparison to a similar nonwoven fabric having a greater percentage of bonded surface area. Nonwoven fabrics, associated systems and methods having such bonding patterns are described in greater detail in copending U.S. Provisional Patent Application No. 63/454,941, filed Mar. 27, 2023 (Published as U.S. Patent Publication No. ______/______), the contents of which are incorporated by reference in its entirety and for all purposes.

In some embodiments, an optional pair of cooperating rolls 120 (also referred to herein as a “press roll”) stabilize the web of fibers by compressing the web before delivery to the winder 118 or the optional bonding unit 116 for bonding. The use of a press roll may be desirable in spunbond manufacturing processes. In some embodiments, for example, the press roll, when present, may include a ceramic coating deposited on a surface thereof. In certain embodiments, for instance, one roll of the pair of cooperating rolls 120 may be positioned above the collection surface 110, and a second roll of the pair of cooperating rolls 120 may be positioned below the collection surface 110. In some embodiments, the system may also include a hot air knife (not shown) that exposes the web 112 to a stream of heated gas, such as air, to lightly bond and stabilize the web.

In some embodiments, the system 100a may further comprise a vacuum source 128 disposed below collection surface 110. Vacuum source 128 provides a vacuum that helps draw and pull the fibers 107 onto collection surface 110.

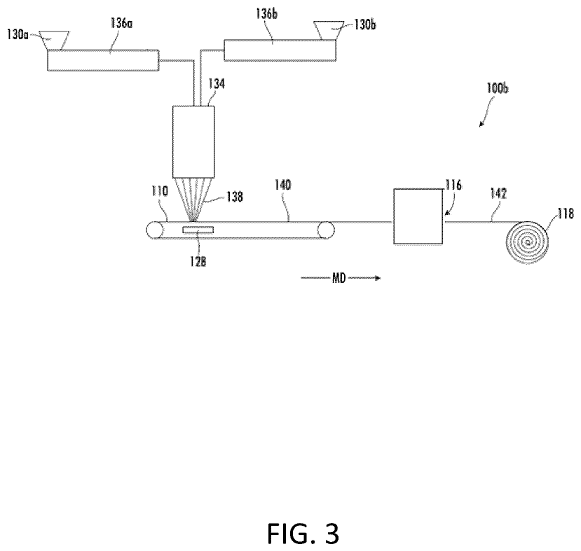

With reference to FIG. 3, a further aspect of a system and method of preparing a nonwoven fabric in accordance with at least one embodiment of the invention is illustrated and broadly designated by reference character 100b. In this embodiment, system 100b may be configured and arranged to produce multicomponent spunbond fibers, such as bicomponent spunbond fibers.

System 100b includes a first polymer source (i.e. hopper) 130a that is in fluid communication with the spunbond spin beam 134 via the extruder 136a. A second polymer source (i.e. hopper) 130b is also in fluid communication with the spunblown spin beam 134 via extruder 136b. In the preparation of multicomponent fabrics, first polymer source may provide a stream of a first polymer resin, and the second polymer source may provide a stream of a second polymer resin. In melt spinning applications, the polymer streams are typically in a molten or semi-molten state. The first polymer resin and the second polymer resin may be different polymers, or may be the same polymers depending on the desired application and desired properties of the nonwoven fabric. For example, the first polymer resin may comprise a first polymer blend comprising a first polypropylene polymer and the elastomeric polyolefin, and the second polymer resin may comprise a second polymer resin, such as a second polypropylene resin or a chemically different type of polymer. In certain embodiments the first polymer resin and the second polymer resin may be the same or different from each other.

Following extrusion, the extruded polymer streams are introduced into the spunbond spin beam 134 at which point the plurality of polymer streams are introduced into a die head (not shown) of a spunbond spin beam. The die head includes a plurality of fluid orifices and an associated cabin system (not shown) for drawing and attenuating the polymer streams as they exit the die head to produce a stream of spunbond fibers.

The spin beam 134 produces a plurality of multicomponent spunbond fibers 138 that are deposited on the collection surface 110 to produce a web 140 of spunbond fibers. At this stage, the spunbond web may comprise a web 140 of multicomponents fibers that are unbonded or slightly bonded to each other.

As discussed above, upon exiting the spin beam, the fibers are introduced into the cabin system in which the fibers are drawn and attenuated. While in the cabin system, the fibers are subjected to cabin pressures in excess of 4,200 Pa to produce fibers having improved fineness and softness.

In certain embodiments, an optional bonding unit 116 is disposed downstream of the collection surface 110 and is configured and arranged to thermally bond filaments to each other to form a coherent web. During thermal bonding the web 140 of fibers, the fibers are heated to a temperature that is sufficient to soften at least one polymer component comprising fibers of web 140 to produce a bonded nonwoven fabric 124. In certain embodiments, the bonded or non-bonded nonwoven fabric moves to a winder 118, where the fabric is then wound onto rolls.

In certain embodiments, the bonding unit comprises an air through bonder in which the fibers are exposed to one or more streams of heated gas, such as air.

In other embodiments, the bonding unit may comprise a calender bonding unit comprising a pair of cooperating heated rolls in which at least one of the rolls includes a plurality of raised bonding points on a surface thereof. As discussed previously, the bonding points can be used to impart a bonding pattern on at least one surface of the nonwoven fabric. In some embodiments, the calender comprises a pair of cooperating rolls in which a first roll comprises an engraved patterned roll having a plurality of bonding points extending from a surface thereof, and the second roll comprises smooth or anvil surface. During bonding, the web of spunbond fibers passes between the pair cooperating rolls, which are heated to a temperature that is sufficient to soften at least one polymer component comprising filaments of web such that the softened polymer component fuses and bonds to adjacent filaments within the web to produce a bonded nonwoven fabric.

As in the previously discussed embodiment, system 100b may also include an optional pair of cooperating rolls (reference character 120 in FIG. 2), optional hot air knife, and vacuum source 128.

Embodiments of the invention, regardless of whether the spunbond system of FIG. 2 or 3 is configured to produce monocomponent or multicomponent fibers, may also include multilayered nonwoven fabrics having 2 to 10 layers, such as 2 to 5, and in particular, 2 to 3 layers. For example, the system may include a first spunbond beam, a meltblown beam, and then a second spunbond beam, wherein each beam deposits a nonwoven fabric layer overlying a previously deposited nonwoven fabric layer. In this example, the system is configured to produce a nonwoven fabric having a spunbond-meltblown-spunbond (SMS) structure.

In other embodiments, system may be configured to prodused a wide variety of different multilayered fabrics including spunbond-spunbond (SS), spunbond-spunbond-meltblown (SSM), spunbond-spunbond-meltblown-spunbond (SSMS), spunbond-spunbond-meltblown-meltblown (SSMM), spunbond-spunbond-meltblown-meltblown-spunbond (SSMMS), spunbond-spunbond-meltblown-meltblown-spunbond-spunbond (SSMMSS), and the like.

Various layers of the nonwoven fabric may include one or more spunbond layers, one or more carded layers, one or more air laid layers, one or more meltblown layers, and the like.

In certain embodiments, the bonded nonwoven fabric may include a layer comprising monocomponent filaments and a second layer comprising multicomponent filaments, such as bicomponent filaments.

In embodiments in which the bonded nonwoven fabric includes multiple layers, the system may include additional fiber forming devices as desired. For example, systems in accordance with embodiments of the invention may include one or more meltblown beams, one or more devices for preparing carded fabric layers, one or more devices for preparing airlaid fabric layers, and the like. Such additional devices may be in the same manufacturing line with the other fiber forming devices to provide a continuous system. Alternatively, one or more additional layers may be provided from a supply roll onto which a previously prepared nonwoven fabric was wound.

In certain embodiments, the nonwoven fabric may include at least one spunbond layer comprising filaments having no crimping or low crimping, and a least one layer comprising crimped filaments.

In accordance with certain embodiments, for instance, bonding the web to form the bonded nonwoven fabric comprises thermal point bonding the web with heat and pressure via a calender having a pair of cooperating rolls including a patterned roll. The patterned roll imparts a three-dimensional geometric bonding pattern onto the nonwoven fabric. Various features of the patterned roll are discussed previously.

In certain embodiments, the inventive nonwoven fabric may be combined with one or more additional nonwoven layers to prepare a composite or laminate material.

As discussed previously, examples of such composites/laminates may include a spunbond composite, such as a spunbond-meltblown (SM) composite, a spunbond-meltblown-spunbond (SMS) composite, or a spunbond-meltblown-meltblown-spunbond (SMMS) composite), a spunbond-spunbond-meltblown-meltblown-spunbond (SSMMS), or a spunbond-spunbond-meltblown-spunbond (SSMS) composite. In some embodiments, composites may be prepared comprising a layer of the bonded nonwoven fabric and one or more film layers. It should be recognized other configurations are also in the scope of the invention.

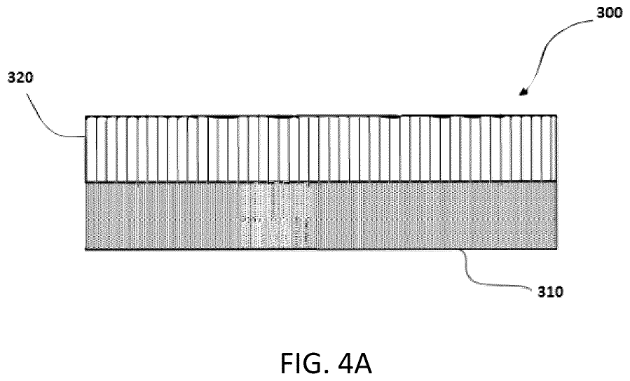

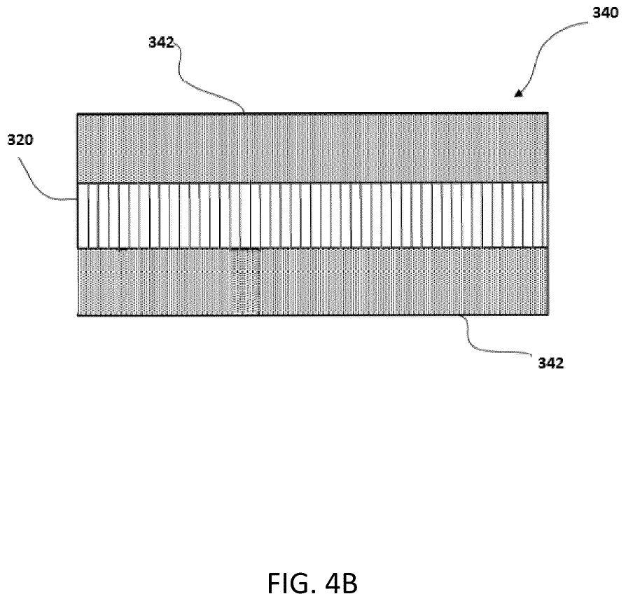

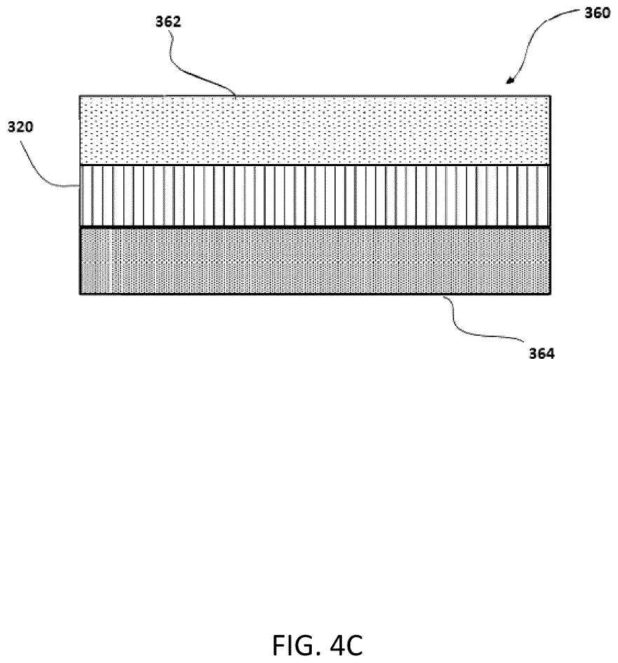

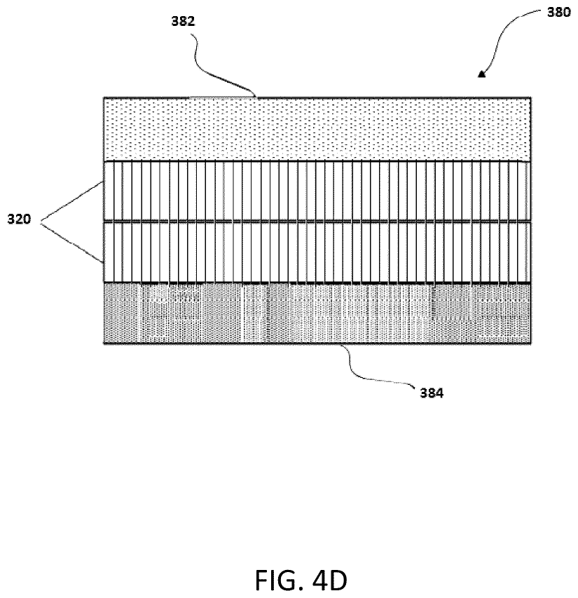

For example, FIGS. 4A-4D are cross-sectional views of composites in accordance with certain embodiments of the invention. For example, FIG. 4A illustrates a spunbond-meltblown (SM) composite 300 having a spunbond nonwoven fabric layer in accordance with embodiments of the present invention 310 and a meltblown layer 320.

FIG. 4B illustrates a spunbond-meltblown-spunbond (SMS) composite 340 having two spunbond nonwoven fabric layers 342 and a meltblown layer 320 sandwiched between the spunbond nonwoven fabric layers 342.

FIG. 4C illustrates an SMS composite 360 having a spunbond nonwoven fabric layer 362, a different spunbond layer 364, and a meltblown layer 320 sandwiched between the two spunbond layers 362, 364.

Finally, FIG. 4D illustrates a spunbond-meltblown-meltblown-spunbond (SMMS) composite 380 having a spunbond nonwoven fabric layer 382, a different spunbond layer 384, and two meltblown layers 320 sandwiched between the two spunbond layers 382, 384. Although the SMMS composite 380 is shown as having two different spunbond layers 382 and 384, both spunbond layers may comprise the same spunbond nonwoven fabric layer, or two different spundbond layers.

In these multilayer structures, the basis weight of the spunbond nonwoven fabric layer may range from as low as 5 g/m² and up to 150 g/m². In some embodiments comprising a multilayer structure (e.g., SM, SMS, and SMMS), the amount of the meltblown in the composite structure may range from about 5 to 30 weight %, and in particular, from about 5 to 15 weight % of the structure as a weight percentage of the structure as a whole.

It should be recognized that the spunbond layers may be the same or different from each other. In addition, the meltblown layers may comprise the same polymer or a different polymer than one or more of the spunbond layers.

In certain embodiments which include a polypropylene meltblown layer, polypropylenes having an MFR that is typically greater than about 500 g/10 min may be used. For example, the polypropylene may have an MFR from about 500 to 2500 g/10 min, and in particular, from about 1000 to 1500 g/10 min, with an MFR from about 1200 to 1400 g/10 min being somewhat more typical. Examples of such a polypropylenes are available from Braskem, such as H155 (1284 MFR) g/10 min.; IPRC Thailand under the product number 1100YC having an MFR of 1,200 g/10 min.; and Lyonedell Basell under the product number HP461Y having an MFR of 1,300 g/10 min.

Multilayer structures in accordance with embodiments can be prepared in a variety of manners including continuous in-line processes where each layer is prepared in successive order on the same line, or depositing a meltblown layer on a previously formed spunbond layer. The layers of the multilayer structure can be bonded together to form a multilayer composite sheet material using thermal bonding, mechanical bonding, adhesive bonding, hydroentangling, or combinations of these.

Multilayer structures in accordance with embodiments can be prepared in a variety of manners including continuous in-line processes where each layer is prepared in successive order on the same line, or depositing a second nonwoven layer on a previously formed spunbond layer. The layers of the multilayer structure can be thermally bonded together to form a multilayer composite sheet material to provide a composite sheet material having the bonding patterns described herein. In addition, composite sheet materials in accordance with certain embodiments of the invention may also be subjected to other bonding techniques, such as thermal bonding via an air through dry, mechanical bonding, adhesive bonding, hydroentangling, or combinations of these. In certain embodiments, the layers may be thermally bonded to each other by passing the multilayer structure through a bonding unit comprising an air through bonder, such as an oven, in the fibers are exposed to a heated stream of gas, such as air.

In some embodiments, the layers may be thermally point bonded to each other by passing the multilayer structure through a bonding unit comprising a pair of calender rolls in which the patterned roll of the calender has an engraved surface comprising a bonding pattern thereon.

In certain embodiments, nonwoven fabrics in accordance with embodiments of the invention may be characterized by having a bonding pattern with a percent bonded area from about 9.6 to 14%, an average individual bond surface area from about 0.10 to 0.25 mm², an average bond point packing value from about 4 to 7.25 mm⁻¹, and one or more of the following properties:

Nonwoven fabrics in accordance with embodiments of the invention may be used to prepare a variety of different structures. For example, in some embodiments, the inventive bonded nonwoven fabric may comprise from about 1 to 10 layers, and in particular, 2 to 8 layers, such as from 3 to 6 layers.

As discussed previously, in certain embodiments, the bonded nonwoven fabric may be combined with one or more additional layers to prepare a composite or laminate material.

Examples of such composites/laminates may include a spunbond composite, such as a spunbond-meltblown (SM) composite, a spunbond-meltblown-spunbond (SMS) composite, or a spunbond-meltblown-meltblown-spunbond (SMMS) composite. In some embodiments, composites may be prepared comprising a layer of the bonded nonwoven fabric and one or more film layers. It should be recognized other configurations are also in the scope of the invention.

As previously noted, fabrics prepared in accordance with embodiments of the invention may be used in wide variety of articles and applications. For instance, embodiments of the invention may be used for personal care applications, for example products for babycare (diapers, wipes), for femcare (pads, sanitary towels, tampons), for adult care (incontinence products), or for cosmetic applications (pads), agricultural applications, for example root wraps, seed bags, crop covers, industrial applications, for example work wear coveralls, airline pillows, automobile trunk liners, sound proofing, and household products, for example mattress coil covers and furniture scratch pads.

The following examples are provided for illustrating one or more embodiments of the present invention and should not be construed as limiting the invention.

### EXAMPLES

Unless otherwise stated, spunbond nonwoven fabrics in the following examples were prepared with a Reicofil 4S spunbond spinning line produced by Reifenhaeuser. Unless otherwise indicated all percentages are weight percentages. The materials and test methods used in the examples are identified below.

### Test Methods:

Basis weight was measured in accordance with NWSP 130.1.

MD and CD tensile strengths were measure in accordance with NWSP 110.4B

(modified: gauge length was 100 mm, sample width was 50 mm, and speed was 100 mm/min.).

MD and CD elongations were measured in accordance with NWSP 110.4B

(modified: gauge length was 100 mm, sample width was 50 mm, and speed was 100 mm/min.).

Air Permeability was measured in accordance with ASTM 90.3.

Hydrohead was measured in accordance with WSP80.6.

Softness Bending was measured in accordance with WSP90.1.

Fiber Fineness (dtex) was measured in accordance with a conventional microscope as is conventional in the art.

### Materials:

“PP-1” refers to a Ziegler-Natta catalyzed homopolymer polypropylene having an MFR of 35 g/10 min available from IPRC Thailand under the product number 1105SC.

“PP-2” refers to a metallocene catalyzed homopolymer polypropylene having an MFR of 30 g/10 min available from Lyondell Basell under the product number HM562S

“PP-3” refers to a meltblown grade polypropylene having an MFR of 1,200 g/10 min available from IPRC Thailand under the product number 1100YC.

“PP-4” refers to a meltblown grade polypropylene having an MFR of 1,300 g/10 min available from Lyonedell Basell under the product number HP461Y.

“PP-5” refers to a metallocene catalyzed polypropylene having an MFR of 25 g/10 min. available from LG under the product number MHE1100

“EPP” refers to an elastomeric polyolefin copolymer having an MFR of 48 g/10 min available from Exxon Mobil under the tradename VISTAMAXX™ 7050FL.

“L-MODU” refers to a low isotacticity polypropylene copolymer are available from Idemitsu under the product name L-MODU™.

“TiO₂” refers to Remafin White PPF2K002G titanium dioxide available from Clariant/Avient under the product code PPON420701.

“GP” refers to a green pigment available from Standridge under the product name SCC Green 0087.

“SA-1” refers to a slip agent available from Evonik Industries under the tradename ACCUREL® SF 617.

“SA-2” refers to a slip agent available from Salee Industries, Thailand under the product name APSA22154.

### Calender Bonding

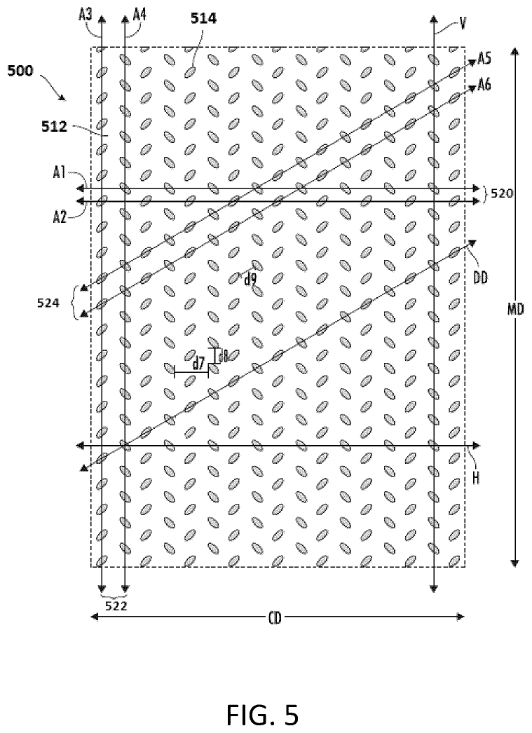

In the following examples, two different calender bonding units were employed. Both calender bonding units had an engraved patterned roll and a corresponding anvil roll with a smooth surface. The general bonding pattern imparted to the surface of the nonwoven fabrics by both engraved pattern rolls is shown in FIG. 5. FIG. 5 illustrates a nonwoven fabric 500 having a bonding pattern on a surface 512 thereof. The bonding pattern generally comprises a plurality of spaced apart bond points 14 that are arranged in a series of alternating arrays that are arranged in pairs 520, 522, 524, of bond points that extend in the machine direction (MD), cross direction (CD), and in the diagonal direction (DD) of the nonwoven fabric. Array pair 520 comprises arrays A1, A2 that extend in the cross direction of the fabric; array pair 522 comprises arrays A3, A4 that extend in the machine direction; and array pair 524 comprises arrays A5, A6 that extend in the diagonal direction of the nonwoven fabric. The arrays extending in the machine direction are substantially aligned (e.g., within) 5° of the vertical axis (V) of the nonwoven fabric, and arrays extending in the machine direction are substantially aligned (e.g., within 5°) of the horizontal axis (H) of the nonwoven fabric.

Calender Bonding Unit 1 provided a bonded nonwoven fabric with a bond density of 49.9 bond points/cm² with each individual bond having a length of 0.882 mm, a width of 0.524 mm, and a bond point surface area of 0.36 mm². The average collective distance between adjacent bond points was 1.19 mm. The bond point distance refers to how closely the bond points are packed together in a given bonding pattern with respect to the machine, cross, and diagonal directions of the nonwoven fabric. The average bond point distance may be calculated from the average of distances between adjacent bonds in the machine direction (FIG. 5, d8), adjacent bonds in the cross direction (FIG. 5, d7), and adjacent bonds in the diagonal direction (FIG. 5, d9) of the nonwoven fabric. The the average bond point packing for the bonding pattern was 3.30 mm⁻¹. The average bond point packing value for a given bonding pattern is calculated by dividing the collective average bond point distance by the average surface area of the bond point for the bonding pattern.

Calender Bonding Unit 2 provided a bonded nonwoven fabric with a bond density of 33.4 bond points/cm² with each individual bond having a length of 1.09 mm, a width of 0.47 mm, and a bond point surface area of 0.4 mm². The average collective distance between adjacent bond points was 1.6 mm. The average bond point packing for the bonding pattern was 4 mm⁻¹.

Calender Bonding Unit 3 provided a bonded nonwoven fabric with a bond pattern having bond density of 55.4 bond points/cm² with each individual bond having a length of 0.76 mm, a width of 0.30 mm, and a bond point surface area of 0.18 mm². The average collective distance between adjacent bond points was 1.28 mm. The average bond point packing for the bonding pattern was 7.11 mm⁻¹.

### Comparative Example 1

In Comparative Example 1, a multilayer layer nonwoven fabric was prepared with a spunbond-meltblown-spunbond (SMS) structure. The spunbond layers comprised filaments composed of a blend of 89.9 weight percent of PP-1 and 0.1 weight percent of TiO₂. The meltblown layer comprised meltblown fibers composed of PP-4. Collectively, the spunbond layers comprised 90 percent by weight of the nonwoven fabric. The spunbond filaments of the spunbond layers were spun at a cabin pressure of approximately 3,300-3,500 Pa with a polymer through put of 2,750 kg/hour.

The extruded filaments were collected on a moving collection surface and the resulting multilayer layer nonwoven fabric was thermally point bonded with Calender Bonding Unit 1. The patterned roll was heated to a temperature of approximately 152° C. The percentage of the surface of the nonwoven fabric bonded in Comparative Example 1 was 18.1%, and the bond density was 49.9 bonding points per cm².

### Inventive Example 1

In Inventive Example 1, a multilayer nonwoven fabric was prepared in a similar manner as in Comparative Example 1 with the exception of die cabin pressure and the polymer composition of the spunbond layers. As in Comparative Example 1, the nonwoven fabric of Inventive Example 1 had an SMS structure. The spunbond layers comprised filaments composed of a blend of. 79.0 weight percent of PP-1, 10 weight percent of EPP, 0.5 weight percent of TiO₂, and 0.5 weight percent of SA-2. The meltblown layer comprised meltblown fibers composed of PP-3. Collectively, the spunbond layers comprised 90 percent by weight of the nonwoven fabric. The spunbond filaments of the spunbond layers were spun at a cabin pressure of approximately 4,500-4,700 Pa with a polymer through put of 2,450 kg/hour.

The multilayer nonwoven fabric of Inventive Example 1 was thermally pointed bonded with Calender Bonding Unit 1 under the same conditions as in Comparative Example 1.

### Comparative Example 2

In Comparative Example 2, the multilayer nonwoven fabric comprised the same SMS structure as in Comparative Example 1. The spunbond layers comprised filaments composed of a blend of 89.9 weight percent of PP-1 and 0.1 weight percent of TiO₂. The meltblown layer comprised meltblown fibers composed of PP-4. Collectively, the spunbond layers comprised 90 percent by weight of the nonwoven fabric. The spunbond filaments of the spunbond layers were spun at a die cabin pressure of approximately 3,300-3,500 Pa with a polymer through put of 2,750 kg/hour.

The nonwoven fabric of Comparative Example 2 was thermally point bonded with Bonding Calender Unit 2. The calender bonding unit was operated at a temperature of approximately 152° C. The percentage of the surface of the nonwoven fabric bonded in Comparative Example 2 was approximately 13.4%, and the bond density was 33.4 bonding points per cm².

### Inventive Example 2

In Inventive Example 2, a multilayer nonwoven fabric was prepared in a similar manner as in Comparative Example 2 with the exception of die cabin pressure and the polymer composition of the spunbond layers. As in Comparative Example 2, the nonwoven fabric of Inventive Example 2 had an SMS structure. The spunbond layers comprised filaments composed of a blend of. 71.4 weight percent of PP-1, 18 weight percent of L-MODU, 0.3 weight percent of TiO₂, and 0.3 weight percent of SA-1. The meltblown layer comprised meltblown fibers composed of PP-4. Collectively, the spunbond layers comprised 90 percent by weight of the nonwoven fabric. The spunbond filaments of the spunbond layers were spun at a cabin pressure of approximately 4,400-4,600 Pa with a polymer through put of 2,450 kg/m/hour.

The multilayer nonwoven fabric of Inventive Example 2 was thermally pointed bonded with same Calender Bonding Unit 2 under the same conditions as in Comparative Example 2.

The nonwoven fabrics of Comparative Examples 1 and 2 and Inventive Examples 1 and 2 were evaluated for mechanical properties, physical properties, softness, and filament fineness. The results are summarized in Table 1, below.

TABLE 1
Comparison of Mechanical Properties

| | Basis | | | MD | CD | | Air |
|---|---|---|---|---|---|---|---|
| | Weight | MD Tensile | CD Tensile | Elongation | Elongation | Hydrohead | Permeability |
| Sample No. | (gsm) | (N/25.4 mm) | (N/25.4 mm) | (%) | (%) | (mm H2O) | L/m²/s |
| Comparative | 15 | 17.3 | 8.3 | 68.2 | 72.0 | 202.80 | 2174 |
| Example 1 | | | | | | | |
| Inventive | 15 | 19.32 | 8.07 | 73.75 | 79.67 | 176.74 | 2191.82 |
| Example 1 | | | | | | | |
| Comparative | 15 | 16.5 | 7.99 | 63.2 | 72.0 | 220.32 | 2500.23 |
| Example 2 | | | | | | | |
| Inventive | 15 | 16.9 | 8.35 | 72.88 | 90.82 | 218.09 | 2507.27d |
| Example 2 | | | | | | | |

The values provided in Table 1 are based on an average of 10 samples.

From Tables 1 and 2, it is evident that the improvements in softness and fiber fineness are accompanied by minor to no reduction in mechanical properties in comparison to an identically prepared fabric with the exception that the identically prepared fabric does not include the elastomeric polyolefin and in which the filaments were drawn and attenuated at a cabin pressure of less than 4,200 Pa. In some embodiments, the inventive nonwoven fabrics have exhibited improvements in both tensile strengths and elongations.

For example, exhibited increases in MD and CD tensile strengths ranging from about 2 to 15% in comparison to an identically prepared fabric with the exception that the identically prepared fabric does not include the elastomeric polyolefin and in which the filaments were drawn and attenuated at a cabin pressure of less than 4,200 Pa. Only Inventive Example 1 had a reduction in mechanical properties (a 2.7% decrease in CD tensile strength in comparison to Comparative Example 1) while all other properties exhibited increases.

Both Inventive Examples 1 and 2 exhibited increases in percent elongations in one or more of machine or cross direction ranging from about 5 to 30% in comparison to Comparative Examples 1 and 2. Notably, Inventive Example 1 exhibited a MD percent elongation increase of 15.3% and a CD percent elongation increase of 26.1 percent.

TABLE 2
Comparison of Fabric Softness and Fiber Fineness

| | | | | Average | |
|---|---|---|---|---|---|
| | Basis | MD | CD | MD/CD | Filament |
| | Weight | Bending | Bending | Bending | Size |
| Sample No. | (gsm) | (mm) | (mm) | (mm) | (dtex) |
| Comparative Example 1 | 15 | 51.84 | 39.07 | 45.45 | 1.76 |
| Inventive Example 1 | 15 | 42.95 | 28.15 | 35.55 | 1.63 |
| Comparative Example 2 | 15 | 50.15 | 39.00 | 44.57 | 1.76 |
| Inventive Example 2 | 15 | 37.94 | 27.80 | 32.87 | 1.3 |

TABLE 3
Percent Decrease and Fiber Fineness Evaluation

| | | | Average | Percent |
|---|---|---|---|---|
| | Percent | | Percent | Decrease |
| | Decrease in | Percent | Decrease in | in |
| | MD | Decrease in | MD/CD | Filament |
| Sample No. | Bending | CD Bending | Bending | size |
| Comparative | 17.2 | 28.0 | 22.6 | 7.4 |
| and Inventive | | | | |
| Examples 1 | | | | |
| Comparative | 24.4 | 28.7 | 26.55 | 26.1 |
| and Inventive | | | | |
| Examples 2 | | | | |

In Table 1, MD and CD tensile strengths between the Comparative Examples and the corresponding Inventive Examples were similar with the Inventive Examples generally exhibiting a modest improvement. Both Inventive Examples 1 and 2 showed increases in elongation in both the MD and CD elongations in comparison to the Comparative Examples.

With respect to Table 2, the Inventive Examples demonstrated improved softness obtained by thinning the fibers during the drawing and attenuation stage. In particular, both inventive examples exhibited a significant decrease in bending length, which means that the nonwoven fabric is softer in comparison to the nonwovens of Comparative Examples 1 and 2 . . . . When viewed in combination with the mechanical processes, it is seen that improvements in softness are obtained by including the elastomeric polyolefin and drawing the filaments under cabin pressures greater than 4,200 Pa.

### Inventive Examples 3 and 4

In Inventive Examples 3 and 4, a three layer spunbond nonwoven fabric was prepared in which each layer comprised the same polymeric blend. The nonwoven fabric was thermally point bonded with Calender Bonding Unit 3 operated at a temperature of approximately 150° C. The percentage of the surface of the nonwoven fabric bonded in Inventive Examples 2 and 3 was 9.92%, and the bond density was 55.4 bonding points per cm². The nonwoven of Inventive Example 3 was prepared at a polymer throughput of 180 kg/m/hr and a cabin pressure of 5,100 Pa. The nonwoven of Inventive Example 4 was prepared at a polymer throughput of 160 kg/m/hr and a cabin pressure of 5,300 Pa.

The fibers of Inventive Examples 3 and 4 comprised a blend of 92.2% PP-2, 7.0% EPP, and 0.8% of TiO₂ and SA-1.

### Inventive Example 5

In the following Inventive Example 5, a three layer spunbond nowoven fabric was prepared with Calender Bonding Unit 2. The system cabin pressure was operated at a pressure above 5,100 Pa with a polymer through put of 200 kg/m/hour.

The fibers of Inventive Example 5 comprised a blend of 81.4% PP-1, 18.0% L-MODU, and 0.8% of TiO₂ and SA. The calender bonding unit was operated at a temperature of approximately 150° C. The percentage of the surface of the nonwoven fabric bonded in Inventive Example 5 was 13.4%, and the bond density was 33.4 bonding points per cm².

The properties of the nonwoven fabric of Inventive Examples 3-5 are provided in the Table 4, below.

TABLE 4
MECHANICAL PROPERTIES OF INVENTIVE EXAMPLES 3-5

| | | | MD | | | | |
|---|---|---|---|---|---|---|---|
| | | | tensile | CD tensile | | | |
| | Basis | Fiber | strength | strength | MD | CD | Air |
| Example | weight | Fineness | (N/25.4 | (N/25.4 | Elongation | Elongation | Permeability |
| No. | (gsm) | (Dtex) | mm) | mm) | (%) | (%) | (m³/m²/s) |
| Inventive | 15 | 1.26 | 17.3 | 7.2 | 58.4 | 76.7 | 83.3 |
| Example 3 | | | | | | | |
| Inventive | 15.1 | 0.9 | 19.3 | 9.1 | 60.4 | 77.4 | 78.1 |
| Example 4 | | | | | | | |
| Inventive | 15 | 1.1-1.3 | 15.2 | 8.6 | 79.9 | 93.5 | 86 |
| Example 5 | | | | | | | |

TABLE 5
BENDING SOFTNESS AND HYDROHEAD
INVENTIVE EXAMPLES 3-5

| | Basis | Fiber | MD | CD | Average | Hydro- |
|---|---|---|---|---|---|---|
| Example | weight | Fineness | Bending | Bending | Bending | head |
| No. | (gsm) | (Dtex) | (mm) | (mm) | (mm) | (mm H₂0) |
| Inventive | 15 | 1.26 | 45.5 | 20.2 | 32.8 | 107.2 |
| Example 3 | | | | | | |
| Inventive | 15.1 | 0.9 | 43.4 | 20.2 | 31.8 | 110.7 |
| Example 4 | | | | | | |
| Inventive | 15 | 1.1-1.3 | 42.1 | 27.9 | 35.0 | 99.9 |
| Example 5 | | | | | | |

Interestingly, it was observed that a reduction in throughput and an increase in cabin pressure resulted in finer fibers as can be seen in comparison of Inventive Examples 2 and 3. In particular, the fibers of Inventive Example 3 exhibited an average filament fineness of 1.26 Dtex whereas the fibers of Inventive Example 4 exhibited an average filament size of 0.9 Dtex. This represent a decrease of 28.6% in fiber fineness.

### Inventive Example 6 (L5 (e-4)

In Inventive Example 6, a nonwoven fabric comprising three spunbond layers was prepared with a Reicofil 5 spunbond spinning line produced by Reifenhaeuser. The three spunbond layers were successively deposited overlying each other in a continuous in-line process.

The fibers were bicomponent fibers having a sheath/core configuration in which the fibers sheath comprised a polymeric blend of 64 weight percent of PP-1, 20 weight percent PP-5, 15.0 weight percent of EPP, 0.5 weight percent TiO₂ and 0.5 weight percent SA-1, and the core comprised 69 weight percent PP-1, 30 weight percent PP-5, 0.5 weight percent TiO₂, and 0.5 weight percent of SA-1. The resulting three layered nonwoven fabric was thermally bonded with Calender Bonding Unit 3. The system cabin pressure was operated a pressures from 4,500-4,700 Pa with a polymer through put of 2,960 kg/hour.

TABLE 6
MECHANICAL PROPERTIES OF INVENTIVE EXAMPLE 6

| | Basis | | | MD | CD | | Air |
|---|---|---|---|---|---|---|---|
| | Weight | MD Tensile | CD Tensile | Elongation | Elongation | Hydrohead | Permeability |
| Sample No. | (gsm) | (N/25.4 mm) | (N/25.4 mm) | (%) | (%) | (mm H2O) | L/m²/s |
| Inventive | 15.1 | 18.8 | 9.0 | 83.8 | 89.0 | 99.6 | 5234.5 |
| Example 6 | | | | | | | |

TABLE 7
Fabric Softness and Fiber Fineness for Inventive Example 6

| | | | | Average | |
|---|---|---|---|---|---|
| | Basis | MD | CD | MD/CD | Filament |
| | Weight | Bending | Bending | Bending | (dtex) |
| Sample No. | (gsm) | (mm) | (mm) | (mm) | Size |
| Inventive | 15.1 | 42.1 | 23.3 | 32.7 | 1.39 |
| Example 6 | | | | | |

Inventive Example 6, which comprised a polymer blend of a metallocene catalyzed polypropylene, a Ziegler-Natta catalyzed polypropylene with the elastomeric polyolefin also exhibited significant improvements in fiber fineness and softness as evidenced by softness bending. In fact, the nonwoven fabric exhibited an average MD/CD bending that was less than Inventive Examples 1 and 2. As shown in Table 6, elongations were also increased.

### Representative Embodiments

The following representative embodiments are provided to highlight features of the disclosed nonwoven fabric. It should be recognized that the representative embodiments may include one or more combinations of following features and also the representative embodiments may not include every provided feature.

In certain embodiments, a nonwoven fabric is provided comprising a plurality of fibers bonded to form a coherent web, the fibers comprising a polymeric blend of a polypropylene resin and an elastomeric polyolefin, wherein the fabric exhibits a decrease in fiber fineness of at least 5% in comparison to an identically prepared nonwoven fabric that does not include the elastomeric polyolefin blended with the polypropylene resin.

In certain such embodiments, the polypropylene resin of the nonwoven fabric has a molecular weight ranging from any of 120,000 to 300,000 g/mol, 140,000 g/mol to about 280,000 g/mol, from about 150,000 to about 250,000 g/mol, and in particular, from about 160,000 to about 180,000 g/mol. In certain embodiments, the polypropylene resin comprises a Ziegler-Natta catalyzed polypropylene, a metallocene catalyzed polypropylene, or a blend thereof. In some embodiments, the polypropylene resin has a melting temperature from about 150° C. to about 175° C.

In certain embodiments of the nonwoven fabric, the polypropylene resin is present in the polymer blend in an amount that is from about 75 to 99 weight percent, based on the total weight of polymer blend, and in particular, from about 80 to 95 weight percent, and more particularly, from about 85 to 94 weight percent, based on the total weight of the polymer blend.

In certain embodiments of the nonwoven fabric, the elasatomeric polypropylene is present in the polymer blend in an amount ranging from about 2 to 30 weight percent, and in particular, 5 to 25 weight percent, and more particularly, from about 8 to 20 weight percent, based on the total weight of the polymer blend.

In certain embodiments, the elastomeric polyolefin is selected from the group consisting of a propylene-alpha-olefin copolymer and a low isotacticity polypropylene polymer. For example, the elastomeric polyolefin comprises a propylene-alpha-olefin copolymer or a low isotacticity polypropylene polymer.

In certain embodiments, elastomeric polyolefin comprises a low isotacticity polypropylene having an isotacticity [mmmm] that is between about 20 and 70% by mol, and in particular, a [mmmm] between 30 and 60% by mol, and more particularly, a [mmmm] between 35 and 55% by mol. In certain embodiments, the low isotacticity polypropylene has the following properties:

an isotacticity: a meso pentad fraction [mmmm] of 20 to 70% by mol;
an average number molecular weight (M_{w}) of 10,000 to 200,000;
a melting temperature from about 60 to 120° C.; and
a melt flow rate (MFR) greater than 40 g/10 min.

In certain embodiments, the nonwoven fabric comprises fibers having an average fiber fineness ranging from about 0.8 to 1.6 Dtex, and in particular, from about 0.9 to 1.4. and more particularly, from about 1.2 to 1.35 Dtex.

In certain embodiments, fibers of the nonwoven fabric were drawn and attenuated at a cabin greater than 4,200 Pa., and the nonwoven fabric exhibits a percent decrease in fiber dtex from about 5 to 30% in comparison to an identically prepared fabric with the exception that the identically prepared fabric does not include the elastomeric polyolefin and is drawn and attenuated at a cabin pressure of less than 4,200 Pa.

In certain embodiments, the nonwoven fabric exhibits a percent decrease in fiber dtex from about 15 to 25% in comparison to an identically prepared fabric with the exception that the identically prepared fabric does not include the elastomeric polyolefin and in which the fibers are drawn and attenuated at a cabin pressure of less than 4,200 Pa.

In certain embodiments, the nonwoven fabric has a machine direction bending softness ranging from 30 to 50 mm and a cross direction bending softness ranging from 15 to 40 mm.

In certain embodiments, the nonwoven fabric has an average machine/cross direction bending softness ranging from 25 to 40 mm, and in particular, from about 28 to 38 mm.

In certain embodiments, the nonwoven fabric fibers were drawn and attenuated at a cabin greater than 4,200 Pa., and the nonwoven fabric exhibits:
a. a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers are drawn and attenuated at a cabin pressure of less than 4,200 Pa,
and
   b. a percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers are drawn and attenuated at a cabin pressure of less than 4,200 Pa.

In certain embodiments, the nonwoven fabric exhibits a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers are drawn and attenuated at a cabin pressure of less than 4,200 Pa.

In certain embodiments, the nonwoven fabric the nonwoven fabric exhibits a percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers are drawn and attenuated at a cabin pressure of less than 4,200 Pa.

In certain embodiments, the nonwoven fabric exhibits one or more of the following characteristics:
a) an average fiber fineness ranging from about 0.8 to 1.6 dtex, and in particular, from about 0.9 to 1.4. and more particularly, from about 1.2 to 1.35 dtex;
b) the nonwoven fabric exhibits a percent decrease in fiber dtex from about 5 to 60%, such as a percent decrease of 15 to 50%, in comparison to an identically prepared fabric with the exception that the identically prepared fabric does not include the elastomeric polyolefin and in which the fibers were drawn and attenuated at a cabin pressure of less than 4,200 Pa;
c) the nonwoven fabric exhibits a machine direction bending softness ranging from 30 to 50 mm direction and a cross direction bending softness ranging from 15 to 40 mm;
d) the nonwoven fabric exhibits a machine direction softness of less than one or more of 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, and 20 mm;
e) a machine direction softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, 44 mm, 46 mm, 48 mm, and 50 mm;
f) an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.
g) a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers were drawn and attenuated at a cabin pressure of less than 4,200 Pa;
h) an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers were drawn and attenuated at a cabin pressure of less than 4,200 Pa;
i) an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the fibers were drawn and attenuated at a cabin pressure of less than 4,200 Pa;
j) an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the fibers were drawn and attenuated at a cabin pressure of less than 4,200 Pa;
k) an increase in MD and CD tensile strengths ranging from about 2 to 15% in comparison to an identically prepared fabric with the exception that the identically prepared fabric does not include the elastomeric polyolefin and in which the fibers were drawn and attenuated at a cabin pressure of less than 4,200 Pa; and
l) an increase in percent elongation in one or more of machine or cross direction ranging from about 5 to 30% in comparison to an identically prepared fabric with the exception that the identically prepared fabric does not include the elastomeric polyolefin and in which the fibers were drawn and attenuated at a cabin pressure of less than 4,200 Pa.

In certain embodiments, the nonwoven fabric has an average fiber fineness ranging from about 0.8 to 1.6 dtex, and in particular, from about 0.9 to 1.4. and more particularly, from about 1.2 to 1.35 dtex.

In certain embodiments, the nonwoven fabric has a percent decrease in fiber dtex from about 5 to 30%, such as a percent decrease of 15 to 25%, in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric exhibits a machine direction bending softness ranging from 30 to 50 mm direction and a cross direction bending softness ranging from 15 to 40 mm, such as a machine direction bending softness ranging from 35 to 45 mm direction and a cross direction bending softness ranging from 25 to 40 mm.

In certain embodiments, the nonwoven fabric has an a machine direction softness of less than one or more of 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, and 20 mm.

In certain embodiments, the nonwoven fabric has an a machine direction bending softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, 44 mm, 46 mm, 48 mm, and 50 mm.

In certain embodiments, the nonwoven fabric has an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.

In certain embodiments, the nonwoven fabric has an a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the filaments were drawn and attenuated at a cabin pressure of less than 4,200 Pa.

In certain embodiments, nonwoven fabric has an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an increase in MD and CD tensile strengths ranging from about 2 to 15% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has an increase in percent elongations in one or more of machine or cross direction ranging from about 5 to 30% in comparison to an identically prepared fabric with the exception that the identically prepared fabric does not include the elastomeric polyolefin and in which the filaments were drawn and attenuated at a cabin pressure of less than 4,200 Pa.

In certain embodiments, the nonwoven fabric has the following:
an average fiber fineness ranging from about 0.8 to 1.6 dtex, and in particular, from about 0.9 to 1.4. and more particularly, from about 1.2 to 1.35 dtex;
a percent decrease in fiber dtex from about 5 to 30%, such as a percent decrease of 15 to 25%, in comparison to an identically prepared fabric with the exception that the identically prepared fabric does not include the elastomeric polyolefin and in which the filaments were drawn and attenuated at a cabin pressure of less than 4,200 Pa; and
a machine direction bending softness ranging from 30 to 50 mm direction and a cross direction bending softness ranging from 15 to 40 mm.

In certain embodiments, the nonwoven fabric comprises a spunbond layer.

In certain embodiments, the fibers of the nonwoven fabric have a sheath/core configuration and wherein the polymer blend comprises the core and a bio-based polymer comprises the sheath. In some embodiments, the bio-based polymer comprises a bio derived polyethylene.

In certain embodiments, the nonwoven fabric comprises a first spunbond layer having low or no crimping filaments and a second layer comprising crimped filaments.

In certain embodiments, the nonwoven fabric comprises at least two layers in which one of the layers is selected from the group of meltblown layer; carded fabric layer, spunbond layer, resin bonded layer, airlaid fabric layer, and a spunlace layer.

In certain embodiments, the nonwoven fabric is in an absorbent article.

In certain embodiments, aspects of the disclosure are directed to a nonwoven article comprising the afore described nonwoven fabric.

Aspects of the invention are also directed to a composite sheet material comprising the afore described nonwoven fabric. In some embodiments, the sheet material comprises a meltblown layer, such as an embodiment in which the meltblown layer is sandwiched between two spunbond layers.

Aspects to the disclosure are directed to a method of preparing a bonded nonwoven fabric comprising:
melt blending a first polypropylene resin and an elastomeric polyolefin to form a molten or semi-molten polymer stream of a polymer blend;
introducing the polymer stream into a spinning beam;
extruding the polymer stream from the spinning beam to form fibers;
subjecting the formed fibers to a cabin pressure greater than 4,200 Pa to draw and attenuate the fibers; and
collecting the fibers on a collection surface to form a nonwoven web, wherein the an average fiber fineness ranging from about 0.8 to 1.6 dtex, and in particular, from about 0.9 to 1.4. and more particularly, from about 1.2 to 1.35 dtex, and the nonwoven fabric exhibits a percent decrease in fiber dtex from about 5 to 30%, such as a percent decrease of 15 to 25%, in comparison to an identically prepared fabric with the exception that the identically prepared fabric does not include the elastomeric polyolefin and in which the fibers were drawn and attenuated at a cabin pressure of less than 4,200 Pa.

In certain embodiments the fibers are drawn and attenuated at a cabin pressure ranges from about 4,500 to 7,500 Pa, and in particular, from about 5,500 to 7,500 Pa.

In certain embodiments of the method, the first polypropylene has a molecular weight ranging from any of 120,000 to 300,000 g/mol, 140,000 g/mol to about 280,000 g/mol, from about 150,000 to about 250,000 g/mol, and in particular, from about 160,000 to about 180,000 g/mol. In some embodiments, the first polypropylene comprises a Ziegler-Natta catalyzed polypropylene, a metallocene catalyzed polypropylene, or a blend thereof. In certain embodiments, the polypropylene resin has a melting temperature from about 150° C. to about 175° C.

In certain embodiments, the first polypropylene is present in the polymer blend in an amount that is from about 75 to 99 weight percent, based on the total weight of polymer blend, and in particular, from about 80 to 95 weight percent, and more particularly, from about 85 to 94 weight percent, based on the total weight of the polymer blend. In certain embodiments, the elasatomeric polypropylene is present in the polymer blend in an amount ranging from about 2 to 30 weight percent, and in particular, 5 to 25 weight percent, and more particularly, from about 8 to 20 weight percent, based on the total weight of the polymer blend.

In certain embodiments, the elastomeric polyolefin comprises a propylene-alpha-olefin copolymer. In certain embodiments, the elastomeric polyolefin comprises a low isotacticity polypropylene polymer.

In certain embodiments, the low isotacticity polypropylene has an isotacticity [mmmm] that is between about 20 and 70% by mol, and in particular, a [mmmm] between 30 and 60% by mol, and more particularly, a [mmmm] between 35 and 55% by mol.

In certain embodiments, the low isotacticity polypropylene has the following properties:
an isotacticity: a meso pentad fraction [mmmm] of 20 to 70% by mol;
an average number molecular weight (M_{w}) of 10,000 to 200,000;
a melting temperature from about 60 to 120° C.; and
a melt flow rate (MFR) greater than 40 g/10 min.

In certain embodiments of the method, the nonwoven fabric comprises fibers having an average fiber fineness ranging from about 0.8 to 1.6 dtex, and in particular, from about 0.9 to 1.4. and more particularly, from about 1.2 to 1.35 dtex.

In certain embodiments of the method, the nonwoven fabric exhibits a percent decrease in fiber dtex from about 5 to 60% in comparison to an identically prepared fabric with the exception that the identically prepared fabric does not include the elastomeric polyolefin and is drawn and attenuated at a cabin pressure of less than 4,200 Pa.

In certain embodiments of the method, the nonwoven fabric exhibits a percent decrease in fiber dtex from about 15 to 60%, such as from 15 to 25%, in comparison to an identically prepared fabric with the exception that the identically prepared fabric does not include the elastomeric polyolefin and in which the fibers are drawn and attenuated at a cabin pressure of less than 4,200 Pa.

In certain embodiments of the method, the nonwoven fabric has a machine direction bending softness ranging from 30 to 50 mm, and a cross direction bending softness ranging from 15 to 40 mm.

In certain embodiments of the method, the nonwoven fabric has an average machine/cross direction bending softness ranging from 25 to 40 mm, and in particular, from about 28 to 38 mm.

In certain embodiments of the method, the nonwoven fabric exhibits a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers are drawn and attenuated at a cabin pressure of less than 4,200 Pa.

In certain embodiments of the method, the nonwoven fabric exhibits a percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers are drawn and attenuated at a cabin pressure of less than 4,200 Pa.

In certain embodiments of the method, the nonwoven fabric exhibits one or more of the following characteristics:
a) an average fiber fineness ranging from about 0.8 to 1.6 dtex, and in particular, from about 0.9 to 1.4. and more particularly, from about 1.2 to 1.35 dtex;
b) a percent decrease in fiber dtex from about 5 to 60%, such as a percent decrease of 15 to 25%, in comparison to an identically prepared fabric with the exception that the identically prepared fabric does not include the elastomeric polyolefin and in which the fibers were drawn and attenuated at a cabin pressure of less than 4,200 Pa;
c) a machine direction bending softness ranging from 30 to 50 mm and a cross direction bending softness ranging from 15 to 40 mm;
d) a machine direction bending softness of less than one or more of mm, 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, and 20 mm;
e) a machine direction softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, 44 mm, 46 mm 48 mm, and 50 mm;
f) an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.
g) a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers were drawn and attenuated at a cabin pressure of less than 4,200 Pa;
h) an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers were drawn and attenuated at a cabin pressure of less than 4,200 Pa;
i) an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the fibers were drawn and attenuated at a cabin pressure of less than 4,200 Pa;
j) an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the fibers were drawn and attenuated at a cabin pressure of less than 4,200 Pa;
k) an increase in MD and CD tensile strengths ranging from about 2 to 15% in comparison to an identically prepared fabric with the exception that the identically prepared fabric does not include the elastomeric polyolefin and in which the fibers were drawn and attenuated at a cabin pressure of less than 4,200 Pa; and
l) an increase in percent elongation in one or more of machine or cross direction ranging from about 5 to 30% in comparison to an identically prepared fabric with the exception that the identically prepared fabric does not include the elastomeric polyolefin and in which the fibers were drawn and attenuated at a cabin pressure of less than 4,200 Pa.

In certain embodiments of the method, the nonwoven fabric has an average fiber fineness ranging from about 0.8 to 1.6 dtex, and in particular, from about 0.9 to 1.4. and more particularly, from about 1.2 to 1.35 dtex.

In certain embodiments of the method, the nonwoven fabric exhibits a percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers are drawn and attenuated at a cabin pressure of less than 4,200 Pa.

In certain embodiments of the method, the nonwoven fabric has a percent decrease in fiber dtex from about 5 to 30%, such as a percent decrease of 15 to 25%, in comparison to an identically prepared fabric with the exception that the identically prepared fabric does not include the elastomeric polyolefin and in which the filaments were drawn and attenuated at a cabin pressure of less than 4,200 Pa.

In certain embodiments of the method, the nonwoven fabric has an a machine direction bending softness ranging from 30 to 50 mm, and a cross direction bending softness ranging from 15 to 40 mm.

In certain embodiments of the method, wherein the nonwoven fabric has an a machine direction softness of less than one or more of 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, and 20 mm.

In certain embodiments of the method, the nonwoven fabric has an a machine direction softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, 44 mm, 46 mm, 48 mm, and 50 mm.

In certain embodiments of the method, the nonwoven fabric has an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.

In certain embodiments of the method, the nonwoven fabric has an a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the filaments were drawn and attenuated at a cabin pressure of less than 4,200 Pa.

In certain embodiments of the method, the nonwoven fabric has an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the filaments were drawn and attenuated at a cabin pressure of less than 4,200 Pa.

In certain embodiments of the method, the nonwoven fabric has an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the filaments were drawn and attenuated at a cabin pressure of less than 4,200 Pa.

In certain embodiments of the method, the nonwoven fabric has an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the filaments were drawn and attenuated at a cabin pressure of less than 4,200 Pa.

In certain embodiments of the method, the nonwoven fabric has an increase in MD and CD tensile strengths ranging from about 2 to 15% in comparison to an identically prepared fabric with the exception that the identically prepared fabric does not include the elastomeric polyolefin and in which the filaments were drawn and attenuated at a cabin pressure of less than 4,200 Pa.

In certain embodiments of the method, the nonwoven fabric has an increase in percent elongations in one or more of machine or cross direction ranging from about 5 to 30% in comparison to an identically prepared fabric with the exception that the identically prepared fabric does not include the elastomeric polyolefin and in which the filaments were drawn and attenuated at a cabin pressure of less than 4,200 Pa.

In certain embodiments of the method, the nonwoven fabric has the following:
an average fiber fineness ranging from about 0.8 to 1.6 dtex, and in particular, from about 0.9 to 1.4. and more particularly, from about 1.2 to 1.35 dtex;
a percent decrease in fiber dtex from about 5 to 360%, such as a percent decrease of 15 to 25%, in comparison to an identically prepared fabric with the exception that the identically prepared fabric does not include the elastomeric polyolefin and in which the filaments were drawn and attenuated at a cabin pressure of less than 4,200 Pa; and
a machine direction bending softness ranging from 30 to 50 mm direction and a cross direction bending softness ranging from 15 to 40 mm, such as a machine direction bending softness ranging from 35 to 45 mm direction and a cross direction bending softness ranging from 25 to 40 mm.

In certain embodiments of the method, the nonwoven web comprises a spunbond web.

In certain embodiments of the method, the method further comprises a step of depositing a second fabric layer over the nonwoven web. In some embodiments, the second fabric layer is selected from the group of meltblown layer; carded fabric layer, spunbond layer, resin bonded layer, airlaid fabric layer, and a spunlace layer.

In certain embodiments of the method, the method further comprises a step of thermal bonding the nonwoven web. In some embodiments, the step of thermal bonding the nonwoven web comprises calender bonding the nonwoven web with an engraved roll having raised bonding points configured and arranged to impart a bonding pattern on a surface of the nonwoven web, the bonding pattern having a percent bonded area from about 9.6 to 14%, an average individual bond surface area from about 0.10 to 0.25 mm², and an average bond point packing value from about 4 to 7.25 mm⁻¹.

Aspects of the disclosure are also directed to the use of the method to prepare an absorbent article.

1. A nonwoven fabric comprising a plurality of fibers bonded to form a coherent web, the fibers comprising a polymeric blend of a polypropylene resin and an elastomeric polyolefin, wherein the fabric exhibits a decrease in fiber fineness of at least 5% in comparison to an identically prepared nonwoven fabric that does not include the elastomeric polyolefin blended with the polypropylene resin.

2. The nonwoven fabric according to claim 1, wherein polypropylene resin has a molecular weight ranging from any of 120,000 to 300,000 g/mol, a melting temperature from about 150° C. to about 175° C., and wherein the polypropylene resin comprises a Ziegler-Natta catalyzed polypropylene, a metallocene catalyzed polypropylene, or a blend thereof.

3. The nonwoven fabric according to claim 1, wherein the elastomeric polypropylene is present in the polymer blend in an amount ranging from about 2 to 30 weight percent, based on the total weight of the polymer blend.

4. The nonwoven fabric according to claim 1, wherein the elastomeric polyolefin is selected from the group consisting of a propylene-alpha-olefin copolymer and a low isotacticity polypropylene polymer.

5. The nonwoven fabric of claim 4, wherein the low isotacticity polypropylene has the following properties:
an isotacticity: a meso pentad fraction [mmmm] of 20 to 70% by mol;
an average number molecular weight (M_{w}) of 10,000 to 200,000;
a melting temperature from about 60 to 120° C.; and
a melt flow rate (MFR) greater than 40 g/10 min.

6. The nonwoven fabric according to claim 1, wherein the nonwoven fabric comprises fibers having an average fiber fineness ranging from about 0.8 to 1.6 Dtex.

7. The nonwoven fabric according to claim 1, wherein the nonwoven fabric exhibits a percent decrease in fiber dtex from about 5 to 30% in comparison to an identically prepared fabric with the exception that the identically prepared fabric does not include the elastomeric polyolefin and is drawn and attenuated at a cabin pressure of less than 4,200 Pa.

8. The nonwoven fabric according to claim 1, wherein the nonwoven fabric has a machine direction bending softness ranging from 30 to 50 mm and a cross direction bending softness ranging from 15 to 40 mm.

9. The nonwoven fabric according to claim 1, wherein the nonwoven fabric has an average machine/cross direction bending softness ranging from 25 to 40 mm, and in particular, from about 28 to 38 mm.

10. The nonwoven fabric according to claim 1, wherein the nonwoven fabric fibers were drawn and attenuated at a cabin greater than 4,200 Pa., and wherein the nonwoven fabric exhibits:
a. a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers are drawn and attenuated at a cabin pressure of less than 4,200 Pa,
and
b. a percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers are drawn and attenuated at a cabin pressure of less than 4,200 Pa.

11. The nonwoven fabric according to claim 1, wherein the nonwoven fabric fibers were drawn and attenuated at a cabin greater than 4,200 Pa., and wherein the nonwoven fabric exhibits one or more of the following characteristics:
a) an average fiber fineness ranging from about 0.8 to 1.6 dtex, and in particular, from about 0.9 to 1.4. and more particularly, from about 1.2 to 1.35 dtex;
b) the nonwoven fabric exhibits a percent decrease in fiber dtex from about 5 to 60%, such as a percent decrease of 15 to 50%, in comparison to an identically prepared fabric with the exception that the identically prepared fabric does not include the elastomeric polyolefin and in which the fibers were drawn and attenuated at a cabin pressure of less than 4,200 Pa;
c) the nonwoven fabric exhibits a machine direction bending softness ranging from 30 to 50 mm direction and a cross direction bending softness ranging from 15 to 40 mm;
d) the nonwoven fabric exhibits a machine direction softness of less than one or more of 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, and 20 mm;
e) a machine direction softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, 44 mm, 46 mm, 48 mm, and 50 mm;
f) an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.
g) a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers were drawn and attenuated at a cabin pressure of less than 4,200 Pa;
h) an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers were drawn and attenuated at a cabin pressure of less than 4,200 Pa;
i) an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the fibers were drawn and attenuated at a cabin pressure of less than 4,200 Pa;
j) an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the fibers were drawn and attenuated at a cabin pressure of less than 4,200 Pa;
k) an increase in MD and CD tensile strengths ranging from about 2 to 15% in comparison to an identically prepared fabric with the exception that the identically prepared fabric does not include the elastomeric polyolefin and in which the fibers were drawn and attenuated at a cabin pressure of less than 4,200 Pa; and
l) an increase in percent elongation in one or more of machine or cross direction ranging from about 5 to 30% in comparison to an identically prepared fabric with the exception that the identically prepared fabric does not include the elastomeric polyolefin and in which the fibers were drawn and attenuated at a cabin pressure of less than 4,200 Pa.

12. The nonwoven fabric according to claim 1, wherein the nonwoven fabric has an increase in MD and CD tensile strengths ranging from about 2 to 15% in comparison to an identically prepared fabric with the exception that the identically prepared fabric does not include the elastomeric polyolefin and in which the filaments were drawn and attenuated at a cabin pressure of less than 4,200 Pa.

13. The nonwoven fabric according to claim 1, wherein the nonwoven fabric has an increase in percent elongations in one or more of machine or cross direction ranging from about 5 to 30% in comparison to an identically prepared fabric with the exception that the identically prepared fabric does not include the elastomeric polyolefin and in which the filaments were drawn and attenuated at a cabin pressure of less than 4,200 Pa.

14. The nonwoven fabric according to claim 1, wherein the nonwoven fabric has the following:
an average fiber fineness ranging from about 0.8 to 1.6 dtex, and in particular, from about 0.9 to 1.4, and more particularly, from about 1.2 to 1.35 dtex;
a percent decrease in fiber dtex from about 5 to 30%, such as a percent decrease of 15 to 25%, in comparison to an identically prepared fabric with the exception that the identically prepared fabric does not include the elastomeric polyolefin and in which the filaments were drawn and attenuated at a cabin pressure of less than 4,200 Pa; and
a machine direction bending softness ranging from 30 to 50 mm direction and a cross direction bending softness ranging from 15 to 40 mm.

15. The nonwoven fabric according to claim 1, wherein the nonwoven fabric comprises a spunbond layer.

16. An absorbent article comprising the nonwoven fabric according to claim 1.

17. Method of preparing a bonded nonwoven fabric comprising:
melt blending a first polypropylene resin and an elastomeric polyolefin to form a molten or semi-molten polymer stream of a polymer blend;
introducing the polymer stream into a spinning beam;
extruding the polymer stream from the spinning beam to form fibers;
subjecting the formed fibers to a cabin pressure greater than 4,200 Pa to draw and attenuate the fibers; and
collecting the fibers on a collection surface to form a nonwoven web, wherein the fibers exhibit an average fiber fineness ranging from about 0.8 to 1.6 dtex, and in particular, from about 0.9 to 1.4. and more particularly, from about 1.2 to 1.35 dtex, and the nonwoven fabric exhibits a percent decrease in fiber dtex from about 5 to 30%, such as a percent decrease of 15 to 25%, in comparison to an identically prepared fabric with the exception that the identically prepared fabric does not include the elastomeric polyolefin and in which the fibers were drawn and attenuated at a cabin pressure of less than 4,200 Pa.

18. The method according to claim 17, wherein the cabin pressure ranges from about 4,500 to 7,500 Pa, and in particular, from about 5,500 to 7,500 Pa.

19. The method according to claim 17, wherein the elastomeric polyolefin comprises a propylene-alpha-olefin copolymer or a low isotacticity polypropylene polymer.

20. The method according to claim 17, wherein the nonwoven fabric exhibits one or more of the following characteristics:
a) an average fiber fineness ranging from about 0.8 to 1.6 dtex, and in particular, from about 0.9 to 1.4. and more particularly, from about 1.2 to 1.35 dtex;
b) a percent decrease in fiber dtex from about 5 to 60%, such as a percent decrease of 15 to 25%, in comparison to an identically prepared fabric with the exception that the identically prepared fabric does not include the elastomeric polyolefin and in which the fibers were drawn and attenuated at a cabin pressure of less than 4,200 Pa;
c) a machine direction bending softness ranging from 30 to 50 mm and a cross direction bending softness ranging from 15 to 40 mm;
d) a machine direction bending softness of less than one or more of mm, 50 mm, 48 mm, 46 mm, 44 mm, 42 mm, 40 mm, and 38 mm, 36 mm, 34 mm, 32 mm, and 30 mm, and a cross direction softness less than one or more of 44 mm, 42 mm, 40 mm, 38 mm, 36 mm, 34 mm, 32 mm, 30 mm, 28 mm, 26 mm, 24 mm, 22 mm, and 20 mm;
e) a machine direction softness of at least one or more of 30 mm, 32 mm, 34 mm, 36 mm, 38 mm, and 40 mm, 42 mm, 46 mm, and 48 mm, and a cross direction softness of at least one or more of 20 mm, least 24 mm, 26 mm, 28 mm, 30 mm, 32 mm, 34 mm, 36 mm, 38 mm, and 40 mm, 42 mm, 44 mm, 46 mm 48 mm, and 50 mm;
f) an average machine/cross direction bending softness ranging from 25 to 40 mm, and in particular, from about 28 to 38 mm, such as an average machine/cross direction bending softness of at least one or more of 20 mm, 22 mm, 24 mm, 26 mm, 28 mm, 30 mm, 32 mm, 34 mm, 36 mm, 38 mm, and 40 mm, and wherein the average machine/cross direction bending softness is less than one or more of 40 mm, 38 mm, 36 mm, 34 mm, 32 mm, 30 mm, 28 mm, 26 mm, 24 mm, 22 mm, and 20 mm.
g) a percent decrease in machine direction bending softness that is from about 20 to 40%, and in particular, from about 25 to 35%, and more particularly, from about 26 to 32% in comparison to an identically prepared fabric with the exception that the identically prepared fabric does not include the elastomeric polyolefin and in which the fibers were drawn and attenuated at a cabin pressure of less than 4,200 Pa;
h) an average percent decrease in machine/cross direction bending softness that is from about 20 to 40%, and in particular, from about 20 to 35%, and more particularly, from about 22 to 30% in comparison to an identically prepared fabric with the exception that the identically prepared fabric does not include the elastomeric polyolefin and in which the fibers were drawn and attenuated at a cabin pressure of less than 4,200 Pa;
i) an average percent decrease in machine/cross direction bending softness that is at least one or more of 12%, 14%, 16%, 18%, 20%, 22%, 24%, 26%, 28%, 30%, 32%, 34%, 36%, 38% and 40% in comparison to an identically prepared fabric with the exception that the identically prepared fabric does not include the elastomeric polyolefin and in which the fibers were drawn and attenuated at a cabin pressure of less than 4,200 Pa;
j) an average percent decrease in machine/cross direction bending softness that is less than one or more of 40%, 38%, 36%, 34%, 32%, 30%, 28%, 26%, 24%, 22%, 20%, 18%, 16%, 14% and 12% in comparison to an identically prepared fabric with the exception that the identically prepared fabric does not include the elastomeric polyolefin and in which the fibers were drawn and attenuated at a cabin pressure of less than 4,200 Pa;
k) an increase in MD and CD tensile strengths ranging from about 2 to 15% in comparison to an identically prepared fabric with the exception that the identically prepared fabric does not include the elastomeric polyolefin and in which the fibers were drawn and attenuated at a cabin pressure of less than 4,200 Pa; and
l) an increase in percent elongation in one or more of machine or cross direction ranging from about 5 to 30% in comparison to an identically prepared fabric with the exception that the identically prepared fabric does not include the elastomeric polyolefin and in which the fibers were drawn and attenuated at a cabin pressure of less than 4,200 Pa.

21. The method according to claim 17, wherein the nonwoven fabric has the following:
an average fiber fineness ranging from about 0.8 to 1.6 dtex, and in particular, from about 0.9 to 1.4. and more particularly, from about 1.2 to 1.35 dtex;
a percent decrease in fiber dtex from about 5 to 360%, such as a percent decrease of 15 to 25%, in comparison to an identically prepared fabric with the exception that the identically prepared fabric does not include the elastomeric polyolefin and in which the filaments were drawn and attenuated at a cabin pressure of less than 4,200 Pa; and
a machine direction bending softness ranging from 30 to 50 mm direction and a cross direction bending softness ranging from 15 to 40 mm, such as a machine direction bending softness ranging from 35 to 45 mm direction and a cross direction bending softness ranging from 25 to 40 mm.

22. The method according to claim 17, further comprising a step of depositing a second fabric layer over the nonwoven web, and wherein the second fabric layer is selected from the group of meltblown layer; carded fabric layer, spunbond layer, resin bonded layer, airlaid fabric layer, and a spunlace layer.

23. The method according to claim 17, further comprising a step of thermal bonding the nonwoven web.

24. The method according to claim 23, wherein the step of thermal bonding the nonwoven web comprises calender bonding the nonwoven web with an engraved roll having raised bonding points configured and arranged to impart a bonding pattern on a surface of the nonwoven web, the bonding pattern having a percent bonded area from about 9.6 to 14%, an average individual bond surface area from about 0.10 to 0.25 mm², and an average bond point packing value from about 4 to 7.25 mm⁻¹.
